SEQUENCE LISTING (9 sequences)

SEQ ID NO: 1 - DNA, 708 bp, Homo sapiens
gtagagatgggggtttcatcctgttggtcaggctggtcttgaactcctgacctcaagtgatctgcctaccttggcctcccaaaaggctgagattacaggcatgagccactgcgccaggccttctttcttttctttttttctttcttttttttttttgagacatcatttagctgtgctgaggggttcttaaataggcagctcagaaaattgttttcctttgtcagccacataaattcagcagaggctcttggagggtccctgctggtgaggggtgaggccagcagtggaactctgatttggtttttgctgagctggtggttgaaaggaatcctactacatcggggttataatagggaagatacattttagaatatgcccagtggagccatcggatgctgcatcgtccccagagagccaagtcatcgtgggccaagctcccatccccatgtctggcctcaactgcaggcccagaatgttgacagctgcctcttggagggttatgggagcctgtgaatgccaacatccccatttgcctgcagcggctgctcccatcctggcttcctggtgggacttttccatgaattggggaatctgctttctgattccaaggcctattaaaatttctgagcattgcccatttcttttgctttatctgtaggacatgggctgtttttaaagaacctcacaaatgaaaaaaaaaaaaaaaaa

SEQ ID NO: 2 - DNA, 25 bp, Homo sapiens
gctttctgattccaaggcctattaa

SEQ ID NO: 3 - DNA, 21 bp, Artificial Sequence
  Description of Artificial Sequence Synthetic oligonucleotide
tccactgggcatattctaaaa

SEQ ID NO: 4 - DNA, 22 bp, Artificial Sequence
  Description of Artificial Sequence Synthetic probe
gtgtaacacgtctatacgccca

SEQ ID NO: 5 - DNA, 24 bp, Artificial Sequence
  Description of Artificial Sequence Synthetic probe
gccttttgggaggccaaggtaggc

SEQ ID NO: 6 - DNA, 60 bp, Artificial Sequence
  Description of Artificial Sequence Synthetic probe
gatgctggtggttggcactcctggcattttccaggacggggttgaaatccctgcggcgtc

SEQ ID NO: 7 - DNA, 25 bp, Artificial Sequence
  Description of Artificial Sequence Synthetic probe
ttaataggccttggaatcagaaagc

SEQ ID NO: 8 - DNA, 25 bp, Artificial Sequence
  Description of Artificial Sequence Synthetic probe
gctttctgattccaaggcctattaa

SEQ ID NO: 9 - protein, 4 aa, Artificial Sequence
  Description of Artificial Sequence Synthetic peptide
DEAD

(19) United States
(12) Patent Application Publication
(10) Pub. No.: US 2013/0136786 A1
(43) Pub. Date: May 30, 2013

(54) LONG NON-CODING RNA SPRY4-IT1 AS A DIAGNOSTIC AND THERAPEUTIC AGENT

(71) Applicant: Ranjan Perera, Orlando, FL (US)

(72) Inventor: Ranjan Perera, Orlando, FL (US)

(73) Assignee: Sanford-Burnham Medical Research Institute

(21) Appl. No.: 13/369,876

(22) Filed: Feb. 9, 2012

Related U.S. Application Data
(60) Provisional application No. 61/441,624, filed on Feb. 10, 2011.

(51) Int. Cl. C12Q 1/68; C12N 15/113

(52) U.S. Cl. 424/450; 514/44 A; 435/6.12; 435/6.11

(57) ABSTRACT

Provided herein are methods for the diagnosis of cancer by comparison of a quantification of long non-coding RNA SPRY4-IT1 with the same measurement taken in a reference sample from a healthy patient. Further provided herein are methods of anticipating the likelihood that such a disease will develop, and methods of treatment in the event of such development.

### CROSS-REFERENCE TO RELATED APPLICATIONS

The application claims benefit of U.S. Provisional Application 61/441,624, filed Feb. 10, 2011.

### FIELD OF THE INVENTION

The present invention relates to methods of diagnosing and treating human cancers.

### BACKGROUND OF THE INVENTION

The following discussion of the background of the invention is merely provided to aid the reader in understanding the invention and is not admitted to describe or constitute prior art to the present invention.

There is considerable interest in understanding the function of RNA transcripts that do not code for proteins in eukaryotic cells. As evidenced by cDNA cloning projects and genomic tiling arrays, more than 90% of the human genome undergoes transcription but does not code for proteins. These transcriptional products are referred to as non-protein coding RNAs (ncRNAs). A variety of ncRNA transcripts, such as ribosomal RNAs, transfer RNAs, and spliceosomal RNAs, are essential for cell function. Similarly, a large number of short ncRNAs such as micro-RNAs (miRNAs), endogenous short interfering RNAs (siRNAs), PIWI-interacting RNAs (piRNAs) and small nucleolar RNAs (snoRNAs) are also known to play important regulatory roles in eukaryotic cells. Recent studies have demonstrated a group of long ncRNA (lncRNA) transcripts that exhibit cell type-specific expression and localize into specific subcellular compartments. lncRNAs are also known to play an important roles during cellular development and differentiation supporting the view that they have been selected during the evolutionary process.

LncRNAs appear to have many different functions. In many cases, they seem to play a role in regulating the activity or localization of proteins, or serve as organizational frameworks for subcellular structures. In other cases, lncRNAs are processed to yield multiple small RNAs or they may modulate how other RNAs are processed. Interestingly, lncRNAs can influence the expression of specific target proteins at specific genomic loci, modulate the activity of protein binding partners, direct chromatin-modifying complexes to their sites of action, and are post-transcriptionally processed to produce numerous 5′-capped small RNAs. Epigenetic pathways can also regulate the differential expression of lncRNAs. lncRNAs are misregulated in various diseases, including ischaemia, heart disease, Alzheimer's disease, psoriasis, and spinocerebellar ataxia type 8. This misregulation has also been shown in various types of cancers, such as breast cancer, colon cancer, prostate cancer, hepatocellular carcinoma and leukemia. One such lncRNA, DD3 (also known as PCA3), is listed as a specific prostate cancer biomarker. Recent studies have revealed the contribution of ncRNAs as proto-oncogenes, e.g. GAGE6, as tumor suppressor genes in tumorigenesis, and as drivers of metastatic transformation, e.g. HOTAIR in breast cancer.

### SUMMARY OF THE INVENTION

The present invention is based on the discovery of the correlation between long non-coding RNA SPRY-IT1 and human cancers, in particular melanoma.

In one aspect, the present invention provides a method for diagnosing melanoma in a subject suspected of having melanoma comprising: (i) assessing the expression level of SPRY4-IT1 in a biological sample obtained from the subject; (ii) comparing the expression level of SPRY4-IT1 in the sample to the a reference expression level derived from the expression level of SPRY4-IT1 in samples obtained from subjects diagnosed as not having melanoma; and (iii) identifying the subject as having melanoma when the expression level of SPRY4-IT1 in the sample is greater than the reference expression level or identifying the subject as not having melanoma when the expression level of SPRY4-IT1 in the sample is not greater than the reference expression level. In some embodiments, the biological sample may comprise skin, skin epidermis, or melanocytes.

In further embodiments, the expression level of SPRY4-IT1 is assessed by evaluating the amount of SPRY4-IT1 mRNA in the biological sample. The evaluation of the SPRY4-IT1 mRNA may, in some embodiments, comprise reverse transcriptase PCR (RT-PCR). The evaluation may further comprise array hybridization, wherein the array comprises an immobilized nucleic acid probe that specifically hybridizes SPRY4-IT1 mRNA, SPRY4-IT1 cDNA, or complements thereof. In still further embodiments, the method may further comprise assessing a SPRY4-IT1 target and identifying the subject as having melanoma when the expression level of both SPRY4-IT1 and the SPRY4-IT1 target is increased. In such cases, the SPRY4-IT1 target may be selected from the group consisting of Ki-67, MCM2, MCM3, MCM4, MCM5, CDK1, CDC20, XIAP, Hsp60, Hsp70, and Livin. In still further embodiments, the method may comprise assessing a SPRY4-IT1 target and identifying the subject as having melanoma when the expression level of SPRY4-IT1 is increased and the expression level of the SPRY4-IT1 target is decreased. In such cases, the SPRY4-IT1 target may be selected from the group consisting of TNFRSF25, DPP-IV, CD26, and Trail R2/DR5.

In another aspect, the present invention provides a method for determining the risk of a subject for developing melanoma comprising; (i) assessing the expression level of SPRY4-IT1 in a biological sample obtained from the subject; (ii) comparing the expression level of SPRY4-IT1 in the sample to the a reference expression level derived from the expression level of SPRY4-IT1 in samples obtained from subjects diagnosed as not having melanoma; and (iii) identifying the subject as having increased risk of developing melanoma when the expression level of SPRY4-IT1 in the sample is greater than the reference expression level or identifying the subject as not having an increased risk of melanoma when the expression level of SPRY4-IT1 in the sample is not greater than the reference expression level. In some embodiments, the biological sample may comprise skin, skin epidermis, or melanocytes.

In further embodiments, the expression level of SPRY4-IT1 is assessed by evaluating the amount of SPRY4-IT1 mRNA in the biological sample. The evaluation of the SPRY4-IT1 mRNA may, in some embodiments, comprise reverse transcriptase PCR(RT-PCR). The evaluation may further comprise array hybridization, wherein the array comprises an immobilized nucleic acid probe that specifically hybridizes SPRY4-IT1 mRNA, SPRY4-IT1 cDNA, or complements thereof. In still further embodiments, the method may further comprise assessing a SPRY4-IT1 target and identifying the subject as having melanoma when the expression level of both SPRY4-IT1 and the SPRY4-IT1 target is increased. In such cases, the SPRY4-IT1 target may be selected from the group consisting of Ki-67, MCM2, MCM3, MCM4, MCM5, CDK1, CDC20, XIAP, Hsp60, Hsp70, and Livin. In still further embodiments, the method may comprise assessing a SPRY4-IT1 target and identifying the subject as having melanoma when the expression level of SPRY4-IT1 is increased and the expression level of the SPRY4-IT1 target is decreased. In such cases, SPRY4-IT1 target may be selected from the group consisting of TNFRSF25, DPP-IV, CD26, and Trail R2/DR5.

In yet another aspect, the present invention provides a method for treating a patient diagnosed as having melanoma comprising administering to the patient an effective amount of a therapeutic agent that reduces SPRY4-IT1 expression. In some embodiments, the SPRY4-IT1 may be reduced in the melanoma cells, and in further embodiments the reduction may be by at least 10%, at least 50%, or at least 90%.

In still another aspect, the present invention provides a method for diagnosing prostate cancer in a subject suspected of having prostate cancer comprising: (i) assessing the expression level of SPRY4-IT1 in a biological sample obtained from the subject; (ii) comparing the expression level of SPRY4-IT1 in the sample to the a reference expression level derived from the expression level of SPRY4-IT1 in samples obtained from subjects diagnosed as not having prostate cancer; and (iii) identifying the subject as having prostate cancer when the expression level of SPRY4-IT1 in the sample is greater than the reference expression level or identifying the subject as not having prostate cancer when the expression level of SPRY4-IT1 in the sample is not greater than the reference expression level.

In yet another aspect, the present invention provides a method for determining the risk of a subject for developing prostate cancer comprising: (i) assessing the expression level of SPRY4-IT1 in a biological sample obtained from the subject; (ii) comparing the expression level of SPRY4-IT1 in the sample to the a reference expression level derived from the expression level of SPRY4-IT1 in samples obtained from subjects diagnosed as not having prostate cancer; and (iii) identifying the subject as having increased risk of developing prostate cancer when the expression level of SPRY4-IT1 in the sample is greater than the reference expression level or identifying the subject as not having an increased risk of prostate cancer when the expression level of SPRY4-IT1 in the sample is not greater than the reference expression level.

In further embodiments of either of the two preceding aspects, the expression level of SPRY4-IT1 is assessed by evaluating the amount of SPRY4-IT1 mRNA in the biological sample. The evaluation of the SPRY4-IT1 mRNA may, in some embodiments, comprise reverse transcriptase PCR (RT-PCR). The evaluation may further comprise array hybridization, wherein the array comprises an immobilized nucleic acid probe that specifically hybridizes SPRY4-IT1 mRNA, SPRY4-IT1 cDNA, or complements thereof. In still further embodiments, the method may further comprise assessing a SPRY4-IT1 target and identifying the subject as having melanoma when the expression level of both SPRY4-IT1 and the SPRY4-IT1 target is increased. In such cases, the SPRY4-IT1 target may be selected from the group consisting of Ki-67, MCM2, MCM3, MCM4, MCM5, CDK1, CDC20, XIAP, Hsp60, Hsp70, and Livin. In still further embodiments, the method may comprise assessing a SPRY4-IT1 target and identifying the subject as having melanoma when the expression level of SPRY4-IT1 is increased and the expression level of the SPRY4-IT1 target is decreased. In such cases, the SPRY4-IT1 target may be selected from the group consisting of TNFRSF25, DPP-IV, CD26, and Trail R2/DR5.

In yet another aspect, the present invention provides a method for treating a patient diagnosed as having prostate cancer comprising administering to the patient an effective amount of a therapeutic agent that reduces SPRY4-IT1 expression. In some embodiments, the SPRY4-IT1 may be reduced in the melanoma cells, and in further embodiments the reduction may be by at least 50%.

The therapeutic agent may be, in further embodiments, an siRNA or an anti-sense nucleic acid, or may comprise a nucleic acid comprising the sequence of SEQ ID NO: 2. The nucleic acid may further be encoded in a vector, which may be a viral vector. The therapeutic agent may additionally be contained within a liposome.

In still a further embodiment, the present invention provides a method for identifying therapeutic agents useful for treating melanoma comprising: (i) providing cells expressing SPRY4-IT1; (ii) treating the cells with a candidate compound; (iii) measuring the expression level of SPRY4-IT1 in the cells after treatment with the candidate compound; and (iv) identifying the candidate compound as useful for treating melanoma when the expression level of SPRY4-IT1 is reduced in the cells relative to the expression level of SPRY4-IT1 in the cells prior to treatment with the candidate compound. In some embodiments, the cells—which may be or be derived from human cells—may comprise melanocytes or melanoma cells.

In further embodiments, the expression level of SPRY4-IT1 is assessed by evaluating the amount of SPRY4-IT1 mRNA in the biological sample. The evaluation of the SPRY4-IT1 mRNA may, in some embodiments, comprise reverse transcriptase PCR (RT-PCR). The evaluation may further comprise array hybridization, wherein the array comprises an immobilized nucleic acid probe that specifically hybridizes SPRY4-IT1 mRNA, SPRY4-IT1 cDNA, or complements thereof.

In another aspect, a method is provided for diagnosing a cancer, the cells of which ectopically express SPRY4-IT1, in a subject suspected of having such cancer, said method comprising: (i) assessing the expression level of SPRY4-IT1 in a biological sample obtained from the subject; (ii) comparing the expression level of SPRY4-IT1 in the sample to the a reference expression level derived from the expression level of SPRY4-IT1 in samples obtained from subjects diagnosed as not having cancer; and (iii) identifying the subject as having cancer when the expression level of SPRY4-IT1 in the sample is greater than the reference expression level or identifying the subject as not having cancer when the expression level of SPRY4-IT1 in the sample is not greater than the reference expression level.

In yet another aspect, a method is provided for determining the risk of a subject for developing a cancer, the cells of which ectopically express SPRY4-IT1, in a subject suspected of being likely to develop such cancer, said method comprising: (i) assessing the expression level of SPRY4-IT1 in a biological sample obtained from the subject; (ii) comparing the expression level of SPRY4-IT1 in the sample to the a reference expression level derived from the expression level of SPRY4-IT1 in samples obtained from subjects diagnosed as not having cancer; and (iii) identifying the subject as having increased risk of developing cancer when the expression level of SPRY4-IT1 in the sample is greater than the reference expression level or identifying the subject as not having an increased risk of cancer when the expression level of SPRY4-IT1 in the sample is not greater than the reference expression level.

In still another aspect, a method is provided for treating a patient diagnosed as having a cancer, the cells of which ectopically express SPRY4-IT1, said method comprising administering to the patient an effective amount of a therapeutic agent that reduces SPRY4-IT1 expression. In some embodiments, the therapeutic agent may further act to down-regulate expression of Ki-67, MCM2, CDK1, CDC20, XIAP, Livin, Hsp60, Hsp70, MCM3, MCM4, or MCM5, or upregulate expression of a gene selected from the group consisting of TNFRSF25, DPP-IV, or Trail R2/DR5. In embodiments of any of the aspects above, the cancer cells may be located in a tumor in an organ selected from the group consisting of the skin, adrenal gland, lung, stomach, testis, prostate, and uterus.

As used herein, the term “nucleic acid molecule” or “nucleic acid” refer to an oligonucleotide, nucleotide or polynucleotide. A nucleic acid molecule may include deoxyribonucleotides, ribonucleotides, modified nucleotides or nucleotide analogs in any combination.

As used herein, the term “nucleotide” refers to a chemical moiety having a sugar (modified, unmodified, or an analog thereof), a nucleotide base (modified, unmodified, or an analog thereof), and a phosphate group (modified, unmodified, or an analog thereof). Nucleotides include deoxyribonucleotides, ribonucleotides, and modified nucleotide analogs including, for example, locked nucleic acids (“LNAs”), peptide nucleic acids (“PNAs”), L-nucleotides, ethylene-bridged nucleic acids (“ENAs”), arabinoside, and nucleotide analogs (including abasic nucleotides).

As used herein, the term “short interfering nucleic acid” or “siNA” refers to any nucleic acid molecule capable of down regulating (i.e., inhibiting) gene expression in a mammalian cells (preferably a human cell). siNA includes without limitation nucleic acid molecules that are capable of mediating sequence specific RNAi, for example short interfering RNA (siRNA), double-stranded RNA (dsRNA), micro-RNA (miRNA), and short hairpin RNA (shRNA).

As used herein, the term “sense region” refers to a nucleotide sequence of a siNA molecule complementary (partially or fully) to an antisense region of the siNA molecule. Optionally, the sense strand of a siNA molecule may also include additional nucleotides not complementary to the antisense region of the siNA molecule.

As used herein, the term “ectopic expression” refers to the occurrence of gene expression or the occurrence of a level of gene expression in a tissue in which it is not generally expressed, or not generally expressed at such a level.

As used herein, the term “SPRY4-IT1 target” refers to a gene coding for a functional biomolecule, i.e., a protein, which is addressed and controlled by SPRY4-IT1. For example, SPRY4-IT1 targets may include, although are not limited to, Ki-67, TNFRSF25, DPP-IV, CD26, MCM2, CDK1, CDC20, XIAP, Hsp60, Hsp70, Trail R2/DR5, MCM3, MCM4, MCM5, and Livin.

As used herein, the term “antisense region” refers to a nucleotide sequence of a siNA molecule complementary (partially or fully) to a target nucleic acid sequence. Optionally, the antisense strand of a siNA molecule may include additional nucleotides not complementary to the sense region of the siNA molecule.

As used herein, the term “duplex region” refers to the region in two complementary or substantially complementary oligonucleotides that form base pairs with one another that allows for a duplex between oligonucleotide strands that are complementary or substantially complementary. For example, an oligonucleotide strand having 21 nucleotide units can base pair with another oligonucleotide of 21 nucleotide units, yet only 19 bases on each strand are complementary or substantially complementary, such that the “duplex region” consists of 19 base pairs. The remaining base pairs may, for example, exist as 5′ and/or 3′ overhangs.

An “abasic nucleotide” conforms to the general requirements of a nucleotide in that it contains a ribose or deoxyribose sugar and a phosphate but, unlike a normal nucleotide, it lacks a base (i.e., lacks an adenine, guanine, thymine, cytosine, or uracil). Abasic deoxyribose moieties include, for example, abasic deoxyribose-3′-phosphate; 1,2-dideoxy-D-ribofuranose-3-phosphate; 1,4-anhydro-2-deoxy-D-ribitol-3-phosphate.

As used herein, the term “inhibit”, “down-regulate”, or “reduce,” with respect to gene expression, means that the level of RNA molecules encoding one or more proteins or protein subunits (e.g., mRNA) is reduced below that observed in the absence of the inhibitor. Expression may be reduced by at least 90%, 80%, 70%, 60%, 50%, 40%, 30%, 20%, 10%, 5% or below the expression level observed in the absence of the inhibitor.

### BRIEF DESCRIPTION OF THE FIGURES

FIG. 1(A) is a genome browser depiction of the SPRY4 locus; (B) and (C) are representations of expression level data of SPRY4-IT1; and (D) is a computational prediction of the secondary structure of SPRY4-IT1.

FIGS. 2(A) and (B) are bar graphs depicting the expression level of SPRY4-IT1 20 human tissues relative to RPLO and SPRY4.1, respectively.

FIG. 3(A)-(D) are bar graphs showing expression of SPRY4-IT1 expression in melanoma patients by location of sample: in primary, nodal metastasis, regional metastasis, and distant metastasis, respectively.

FIGS. 4(A) and (B) are bar graphs showing expression of SPRY4-IT1 following knockdown by siRNA; (C) is a photograph of the gel confirming the occurrence of the knockdown; and (D) is a series of photographs showing localization of SPRY4-IT1 in melanocytes.

FIGS. 5(A) and (B) are graphs showing viability of melanoma cells; (C) shows the results of flow cytometry after SPRY4-IT1 knockdown; (D) is a graph showing invasion potential, and (E) is a series of photographs of invading cells.

FIG. 6 is a data cluster of the microarray data from normal and melanoma patient skin samples.

FIG. 7(A)-(D) is cDNA sequencing data illustrating expression levels of four melanoma-specific lncRNAs.

FIG. 8 is a graph of qRT-PCR results for SPRY4-IT1 expression levels in four types of cells.

FIG. 9(A)-(D) is sequencing data showing mapped tag densities for SPRY1, SPRY2, SPRY3, and SPRY4 loci, respectively.

FIG. 10 is a bar graph showing quantitative mRNA levels of SPRY4 in melanoma and melanocytes.

FIG. 11(A)-(C) are bar graphs showing the expression of the two SPRY4 alternate mRNA isoforms in 20 normal human tissues.

FIG. 12(A)-(D) are bar graphs showing relative expression of SPRY4-IN-1 to SPRY4.2 in primary, nodal metastasis, regional metastasis, and distant metastasis samples, respectively.

FIG. 13 is a bar graph showing SPRY4 expression in a dose-dependent knockdown of SPRY4-IT1 in melanoma.

FIG. 14 is the cDNA nucleotide sequence for SPRY4-IT1 (GenBank Accession No. AK024556; SEQ ID NO: 1).

FIG. 15 is a series of photographs of melanocytes infected with control lenti-vector and lenti-SPRY4-IT1 vector stained with texas red, GFP, DAPI, and Merged. As shown, SPRY4-IT1 is primarily transported into the cytoplasm in cells engineered to ectopically express SPRY4-IT1.

FIG. 16 is a line graph showing activation of melanocyte proliferation by infection with SPRY4-IT1 over time in control lenti-vector infected cells and lenti-SPRY4-IT1-infected cells.

FIG. 17 is a series of photographs showing proliferation of cells engineered to ectopically express SPRY4-IT1 as compared to control cells.

FIG. 18 is a bar graph showing the relative mRNA levels of target genes of SPRY4-IT1 as expressed in qRT-PCR.

FIG. 19 is a diagram illustration the cloning strategy for the SPRY4-IT1 upstream sequence and entire SPRY4 intron 1.

FIG. 20 is a bar graph showing SPRY4-IT1 putative promoter expression via luciferase expression containing the putative promoter construct (pcDNA/Luc/SP-IT1) and controls.

FIG. 21 is a line graph demonstrating the rate of decay of RNA of SPRY4-IT1 compared to its host gene after treatment with α-Amanitin.

FIG. 22 is a series of bar graphs showing the expression of SPRY4-IT1 in tumor cells from various organs (A), the adrenal gland (C), the lung (E) and the log 2 expression of the same (B), (D), (F).

FIG. 23 is a series of bar graphs showing the expression of SPRY4-IT1 in tumor cells from the stomach (A), testis (C), and uterus (E), and the log 2 expression of the same (B), (D), (F).

FIG. 24 is a series of photographs showing expression of Ki-67 in melanocytes expressing SPRY4-IT1 as compared to cells expressing empty vector.

### DETAILED DESCRIPTION

The present invention relates generally to identifying and characterizing long non-coding RNAs (“lncRNAs”) that are differentially expressed in cancer cells, particularly in melanoma, as compared to melanocytes or normal skin. In particular, one such lncRNA, SPRY4-IT1, located in the intronic region of the SPRY4 gene, has been shown to be unregulated in melanoma cells and in tumor cells found in the stomach, the adrenal gland, the uterus, the testis, and the lung. SPRY4 is an inhibitor of the receptor-transduced mitogen-activated protein kinase (MAPK) signaling pathway that functions upstream of RAS activation and impairs the formation of active GTP-RAS. Downregulation of the expression of SPRY4-IT1 results in defects in cell growth, differentiation and elevated rates of apoptosis in melanoma cells.

The identification of cancer-associated lncRNAs and the investigation of their molecular and biological functions aids in understanding the molecular etiology of cancer and its progression. Data provided herein demonstrates that a number of lncRNAs are differentially expressed in melanoma cell lines in comparison to melanocytes and keratinocyte controls. One of these lncRNAs, SPRY4-IT1 (Genbank accession ID AK024556), is derived from an intron of the SPRY4 gene and is predicted to contain several long hairpins in its secondary structure. RNA-FISH analysis demonstrates that SPRY4-IT1 is predominantly accumulated in melanoma cell cytoplasm, and SPRY4-IT1 knock-down by stealth siRNAi results in defects in cell growth, differentiation and higher rates of apoptosis in melanoma cell lines. Differential expression of both SPRY4 and SPRY4-IT1 was also detected in vivo, in 30 distinct patient samples, classified as primary in situ, regional metastatic, distant metastatic, and nodal metastatic melanoma. The elevated expression of SPRY4-IT1 in melanoma cells compared to melanocytes, its accumulation in cell cytoplasm, and effects on cell dynamics demonstrates that SPRY4-IT1 plays an important role in human melanoma.

Sprouty (SPRY) is a Ras/Erk inhibitor protein and there are four SPRY genes (SPRY1, SPRY2, SPRY3 and SPRY4) in human, SPRY4 which is the host gene of lncRNA SPRY4-IT1, occurs in two alternately spliced isoforms, termed SPRY4.1 and SPRY4.2, the latter of which retains an additional exon that results in translation initiating from an alternate start codon. To better understand where SPRY4 functions and the relative, expression of the two isoforms, qRT-PCR was used to measure the expression of SPRY4.1 and SPRY4.2 across 20 human tissues. Differential expression levels of these isoforms indicate that the existence of an isoform specific regulatory mechanisms in melanomas and normal human tissues. Deep-sequencing results show that SPRY1 and SPRY3 have little or no expression in both melanoma and melanocytes, but SPRY2 and SPRY4 are highly expressed in melanoma cells compared to melanocytes. Preliminary results indicate, however, that SPRY-IT1 regulation is independent of its master gene, SPRY4.

SPRY4-IT1 is expressed in melanoma cells but not in melanocytes. The elevated expression of SPRY4-IT1 in melanoma cells compared to melanocytes, its accumulation in cell cytoplasm, and effects on cell dynamics suggest that SPRY4-IT1 plays an important role in melanoma development and is an early biomarker and a key regulator for melanoma pathogenesis in human.

SPRY4-IT1 is also shown herein to be expressed in tumor cells of organs other than the skin, including, for example, adrenal gland, lung, stomach, testis, prostate, and uterus.

Several targets of SPRY4-IT1 are identified herein. Those targets include the cell proliferation protein Ki-67, the pro-apoptotic gene TNERSF25, DPP-IV, a cell surface protein that suppresses development of melanoma, MCM2, MCM3, MCM4, and MCM5, which code for DNA replication licensing factor, CDK1, which acts as a serine/threonine kinase and is a key player in cell cycle regulation, CDC20, which regulates cell division, Xiap, or x-linked inhibitor of apoptotis protein, Livin, another anti-apoptotic gene, Hsp60 and Hsp70, heat shock proteins responsible for responsible for the transportation and refolding of proteins from the cytoplasm into the mitochondrial matrix, Trail R2/DR5, an anti-inflammatory cytokine, and rck/p54, a DEAD box protein that has been shown to be overexpressed in colorectal cancers.

RNA Interference and siNA

RNA interference refers to the process of sequence-specific post-transcriptional gene silencing in animals mediated by short interfering RNAs (siRNAs) (Zamore et al., 2000, Cell, 101, 25-33; Fire et al., 1998, Nature, 391, 806; Hamilton et al., 1999. Science, 286, 950-951; Lin et al., 1999, Nature, 402, 128-129; Sharp, 1999, Genes & Dev., 13:139-141; and Strauss, 1999, Science, 286, 886). Post-transcriptional gene silencing is believed to be an evolutionarily-conserved cellular mechanism for preventing expression of foreign genes that may be introduced into the host cell (Fire et al., 1999, Trends Genet., 15, 358). Post-transcriptional gene silencing may be an evolutionary response to the production of double-stranded RNAs (dsRNAs) resulting from viral infection or from the random integration of transposable elements (transposons) into a host genome. The presence of dsRNA in cells triggers the RNAi response that appears to be different from other known mechanisms involving double stranded RNA-specific ribonucleases, such as the interferon response that results from dsRNA-mediated activation of protein kinase PKR and 2′,5′-oligoadenylate synthetase resulting in non-specific cleavage of mRNA by ribonuclease L (see for example U.S. Pat. Nos. 6,107,094; 5,898,031; Clemens et al., 1997, J. Interferon & Cytokine Res., 17, 503-524; Adah et al., 2001, Curr. Med. Chem., 8, 1189).

The presence of long dsRNAs in cells stimulates the activity of dicer, a ribonuclease III enzyme (Bass, 2000, Cell, 101, 235; Zamore et al., 2000, Cell, 101, 25-33; Hammond et al. 2000, Nature, 404, 293). Dicer processes long dsRNA into double-stranded short interfering RNAs (siRNAs) which are typically about 21 to about 23 nucleotides in length and include about 19 base pair duplexes (Zamore et al., 2000, Cell, 101, 25-33; Bass, 2000, Cell, 101, 235; Elbashir et al., 2001, Genes Dev., 15, 188).

Single-stranded RNA, including the sense strand of siRNA, trigger an RNAi response mediated by an endonuclease complex known as an RNA-induced silencing complex (RISC). RISC mediates cleavage of this single-stranded RNA in the middle of the siRNA duplex region (i.e., the region complementary to the antisense strand of the siRNA duplex) (Elbashir et al., 2001, Genes Dev., 15, 188).

In certain embodiments, the siNAs may be a substrate for the cytoplasmic Dicer enzyme (i.e., a “Dicer substrate”) which is characterized as a double stranded nucleic acid capable of being processed in vivo by Dicer to produce an active nucleic acid molecules. The activity of Dicer and requirements for Dicer substrates are described, for example, U.S. 2005/0244858. Briefly, it has been found that dsRNA, having about 25 to about 30 nucleotides, effective result in a down-regulation of gene expression. Without wishing to be bound by any theory, it is believed that Dicer cleaves the longer double stranded nucleic acid into shorter segments and facilitates the incorporation of the single-stranded cleavage products into the RNA-induced silencing complex (RISC complex). The active RISC complex, containing a single-stranded nucleic acid cleaves the cytoplasmic RNA having complementary sequences.

It is believed that Dicer substrates must conform to certain general requirements in order to be processed by Dicer. The Dicer substrates must of a sufficient length (about 25 to about 30 nucleotides) to produce an active nucleic acid molecule and may further include one or more of the following properties: (i) the dsRNA is asymmetric, e.g., has a 3′ overhang on the first strand (antisense strand) and (ii) the dsRNA has a modified 3′ end on the antisense strand (sense strand) to direct orientation of Dicer binding and processing of the dsRNA to an active siRNA. The Dicer substrates may be symmetric or asymmetric. For example, Dicer substrates may have a sense strand includes 22-28 nucleotides and the antisense strand may include 24-30 nucleotides, resulting in duplex regions of about 25 to about 30 nucleotides, optionally having 3′-overhangs of 1-3 nucleotides.

Dicer substrates may have any modifications to the nucleotide base, sugar or phosphate backbone as known in the art and/or as described herein for other nucleic acid molecules (such as siNA molecules).

The RNAi pathway may be induced in mammalian and other cells by the introduction of synthetic siRNAs that are 21 nucleotides in length (Elbashir et al., 2001, Nature, 411, 494 and Tuschl et al., WO 01/75164; incorporated by reference in their entirety). Other examples of the requirements necessary to induce the down-regulation of gene expression by RNAi are described in Zamore et al., 2000, Cell, 101, 25-33; Bass, 2001, Nature, 411, 428-429; Kreutzer et al., WO 00/44895; Zernicka-Goetz et al., WO 01/36646; Fire, WO 99/32619; Plaetinck et al., WO 00/01846; Mello and Fire, WO 01/29058; Deschamps-Depaillette, WO 99/07409; and Li et al., WO 00/44914; Allshire, 2002, Science, 297. 1818-1819; Volpe et al., 2002, Science, 297, 1833-1837; Jenuwein, 2002, Science, 297, 2215-2218; and Hall et al., 2002, Science, 297, 2232-2237: Hutvagner and Zamore, 2002, Science, 297, 2056-60; McManus et al., 2002, RNA, 8, 842-850; Reinhart et al., 2002, Gene & Dev., 16, 1616-1626; and Reinhart & Bartel, 2002, Science, 297. 1831; each of which is hereby incorporated by reference in its entirety.

Briefly, an siNA nucleic acid molecule can be assembled from two separate polynucleotide strands (a sense strand and an antisense strand) that are at least partially complementary and capable of forming stable duplexes. The length of the duplex region may vary from about 15 to about 49 nucleotides (e.g., about 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, or 49 nucleotides). Typically, the antisense strand includes nucleotide sequence that is complementary to nucleotide sequence in a target nucleic acid molecule. The sense strand includes nucleotide sequence corresponding to the target nucleic acid sequence which is therefore at least substantially complementary to the antisense stand. Optionally, an siNA is “RISC length” and/or may be a substrate for the Dicer enzyme. Optionally, an siNA nucleic acid molecule may be assembled from a single polynucleotide, where the sense and antisense regions of the nucleic acid molecules are linked such that the antisense region and sense region fold to form a duplex region (i.e., forming a hairpin structure).

### 5′ Ends, 3′ Ends and Overhangs

siNAs may be blunt-ended on both sides, have overhangs on both sides or a combination of blunt and overhang ends. Overhangs may occur on either the 5′- or 3′-end of the sense or antisense strand. Overhangs typically consist of 1-8 nucleotides (e.g., 1, 2, 3, 4, 5, 6, 7, or 8 nucleotides each) and are not necessarily the same length on the 5′- and 3′-end of the siNA duplex. The nucleotide(s) forming the overhang need not be of the same character as those of the duplex region and may include deoxyribonucleotide(s), ribonucleotide(s), natural and non-natural nucleobases or any nucleotide modified in the sugar, base or phosphate group such as disclosed herein.

The and/or 3′-end of one or both strands of the nucleic acid may include a free hydroxyl group or may contain a chemical modification to improve stability. Examples of end modifications (e.g., terminal caps) include, but are not limited to, abasic, deoxy abasic, inverted (deoxy) abasic, glyceryl, dinucleotide, acyclic nucleotide, amino, fluoro, chloro, bromo, CN, CF, methoxy, imidazole, carboxylate, thioate, C1 to C10 lower alkyl, substituted lower alkyl, alkaryl or aralkyl, OCF3, OCN, O-, S-, or N-alkyl; O-, S-, or N-alkenyl; SOCH3; SO2CH3; ONO2; NO2, N3; heterocycloalkyl; heterocycloalkaryl, aminoalkylamino; polyalkylamino or substituted silyl, as, among others, described in European patents EP 586,520 and FP 618,925.

### Chemical Modifications

siNA molecules optionally may contain one or more chemical modifications to one or more nucleotides. There is no requirement that chemical modifications are of the same type or in the same location on each of the siNA strands. Thus, each of the sense and antisense strands of an siNA may contain a mixture of modified and unmodified nucleotides. Modifications may be made for any suitable purpose including, for example, to increase RNAi activity, increase the in vivo stability of the molecules (e.g., when present in the blood), and/or to increase bioavailability.

Suitable modifications include, for example, internucleotide or internucleoside linkages, dideoxyribonucleotides, 2′-sugar modification including amino, fluoro, methoxy, alkoxy and alkyl modifications; 2′-deoxyribonucleotides, 2′-O-methyl ribonucleotides, 2′-deoxy-2′-fluoro ribonucleotides, “universal base” nucleotides, “acyclic” nucleotides, 5-C-methyl nucleotides, biotin group, and terminal glyceryl and/or inverted deoxy abasic residue incorporation, sterically hindered molecules, such as fluorescent molecules and the like. Other nucleotides modifiers could include 3′-deoxyadenosine (cordycepin), 3′-azido-3′-deoxythymidine (AZT), 2′,3′-dideoxyinosine (ddI), 2′,3′-dideoxy-3′-thiacytidine (3TC), 2′,3′-didehydro-2′,3′-dideoxythymidine (d4T) and the monophosphate nucleotides of 3′-azido-3′-deoxythymidine (AZT), 2′,3′-dideoxy-3′-thiacytidine (3TC) and 2′,3′-didehydro-2′,3′-dideoxythymidine (d4T).

Other suitable modifications include, for example, locked nucleic acid (LNA) nucleotides (e.g., 2′-O, 4′-C-methylene-(D-ribofuranosyl) nucleotides); 2′-methoxyethoxy (MOE) nucleotides; 2′-methyl-thio-ethyl, 2′-deoxy-2′-fluoro nucleotides, 2′-deoxy-2′-chloro nucleotides, 2′-azido nucleotides, and 2′-O-methyl nucleotides (WO 00/47599, WO 99/14226, WO 98/39352, and WO 2004/083430).

Chemical modifications also include terminal modifications on the 5′ and/or 3′ part of the oligonucleotides and are also known as capping moieties. Such terminal modifications are selected from a nucleotide, a modified nucleotide, a lipid, a peptide, and a sugar.

Chemical modifications also include L-nucleotides. Optionally, the L-nucleotides may further include at least one sugar or base modification and/or a backbone modification as described herein.

### Delivery of Nucleic Acid-Containing Pharmaceutical Formulations

Nucleic acid molecules disclosed herein (including siNAs and Dicer substrates) may be administered with a carrier or diluent or with a delivery vehicle which facilitate entry to the cell. Suitable delivery vehicles include, for example, viral vectors, viral particles, liposome formulations, and lipofectin.

Methods for the delivery of nucleic acid molecules are described in Akhtar et al., Trends Cell Bio., 2: 139 (1992); Delivery Strategies for Antisense Oligonucleotide Therapeutics, ed. Akhtar, (1995). Maurer et al., Mol. Membr. Biol., 16: 129-140 (1999); Hofland and Huang, Handb. Exp. Pharmacol., 137: 165-192 (1999); and Lee et al., ACS Symp. Ser., 752: 184-192 (2000); U.S. Pat. Nos. 6,395,713; 6,235,310; 5,225,182; 5,169,383; 5,167,616; 4,959,217; 4,925,678; 4,487,603; and 4,486,194; WO 94/02595; WO 00/03683; WO 02/08754; and U.S. 2003/077829.

Nucleic acid molecules can be administered to cells by a variety of methods known to those of skill in the art, including, but not restricted to, encapsulation in liposomes, by iontophoresis, or by incorporation into other vehicles, such as biodegradable polymers, hydrogels, cyclodextrins (see e.g., Gonzalez et al., Bioconjugate Chem., 10: 1068-1074 (1999); WO 03/47518; and WO 03/46185), poly(lactic-co-glycolic)acid (PLGA) and PLCA microspheres (see for example U.S. Pat. No. 6,447,796 and U.S. 2002/130430), biodegradable nanocapsules, and bioadhesive microspheres, or by proteinaceous vectors (WO 00/53722). Alternatively, the nucleic acid/vehicle combination is locally delivered by direct injection or by use of an infusion pump. Direct injection of the nucleic acid molecules of the invention, whether subcutaneous, intramuscular, or intradermal, can take place using standard needle and syringe methodologies, or by needle-free technologies such as those described in Conry et al., Clin. Cancer Res., 5: 2330-2337 (1999) and WO 99/31262. The molecules of the instant invention can be used as pharmaceutical agents.

Nucleic acid molecules may be complexed with cationic lipids, packaged within liposomes, or otherwise delivered to target cells or tissues. The nucleic acid or nucleic acid complexes can be locally administered to relevant tissues ex vivo, or in vivo through direct dermal application, transdermal application, or injection, with or without their incorporation in biopolymers. Delivery systems include surface-modified liposomes containing poly(ethylene glycol) lipids (PEG-modified, or long-circulating liposomes or stealth liposomes).

Nucleic acid molecules may be formulated or complexed with polyethylenimine (e.g., linear or branched PEI) and/or polyethylenimine derivatives, including for example polyethyleneimine-polyethyleneglycol-N-acetylgalactosamine (PEI-PEG-GAL) or polyethyleneimine-polyethyleneglycol-tri-N-acetylgalactosamine (PEI-PEG-triGAL) derivatives, grafted PEIs such as galactose PEI, cholesterol PEI, antibody derivatized PEI, and polyethylene glycol PEI (PEG-PEI) derivatives thereof (see, for example Ogris et al., 2001, AAPA PharmSci, 3, 1-11; Furgeson et al., 2003, Bioconjugate Chem., 14, 840-847; Kunath et al., 2002, Pharmaceutical Research, 19, 810-817; Choi et al., 2001, Bull. Korean Chem. Soc., 22, 46-52; Bettinger et al., 1999, Bioconjugate Chem., 10, 558-561; Peterson et al., 2002, Bioconjugate Chem., 13, 845-854; Erbacher et al., 1999, Journal of Gene Medicine Preprint, 1, 1-18; Godbey et al., 1999, PNAS USA, 96, 5177-5181; Godbey et al., 1999, Journal of Controlled Release, 60, 149-160; Diebold et al., 1999, Journal of Biological Chemistry, 274, 19087-19094; Thomas and Klibanov, 2002, PNAS USA, 99, 14640-14645; U.S. Pat. No. 6,586,524 and U.S. 2003/0077829).

Delivery systems may include, for example, aqueous and nonaqueous gels, creams, multiple emulsions, microemulsions, liposomes, ointments, aqueous and nonaqueous solutions, lotions, aerosols, hydrocarbon bases and powders, and can contain excipients such as solubilizers, permeation enhancers (e.g., fatty acids, fatty acid esters, fatty alcohols and amino acids), and hydrophilic polymers (e.g., polycarbophil and polyvinylpyrolidone). In one embodiment, the pharmaceutically acceptable carrier is a liposome or a transdermal enhancer. Examples of liposomes which can be used in this invention include the following: (1) CellFectin, 1:1.5 (M/M) liposome formulation of the cationic lipid N,NI,NII,NIII-tetramethyl-N,NI,NII,NIII-tetrapalmit-y-spermine and dioleoyl phosphatidylethanolamine (DOPE) (GIBCO BRL); (2) Cytofectin GSV, 2:1 (M/M) liposome formulation of a cationic lipid and DOPE (Glen Research); (3) DOTAP (N-[1-(2,3-dioleoyloxy)-N,N,N-tri-methyl-ammoniummethylsulfate) (Boehringer Manheim); and (4) Lipofectamine, 3:1 (M/M) liposome formulation of the polycationic lipid DOSPA, the neutral lipid DOPE (GIBCO BRL) and Di-Alkylated Amino Acid (DiLA2).

Therapeutic nucleic acid molecules may be expressed from transcription units inserted into DNA or RNA vectors. Recombinant vectors can be DNA plasmids or viral vectors. Nucleic acid molecule expressing viral vectors can be constructed based on, but not limited to, adeno-associated virus, retrovirus, adenovirus, or alphavirus. The recombinant vectors are capable of expressing the nucleic acid molecules either permanently or transiently in target cells. Delivery of nucleic acid molecule expressing vectors can be systemic, such as by intravenous, subcutaneous, or intramuscular administration.

Expression vectors may include a nucleic acid sequence encoding at least one nucleic acid molecule disclosed herein, in a manner which allows expression of the nucleic acid molecule. For example, the vector may contain sequence(s) encoding both strands of a nucleic acid molecule that include a duplex. The vector can also contain sequence(s) encoding a single nucleic acid molecule that is self-complementary and thus forms a nucleic acid molecule. Non-limiting examples of such expression vectors are described in Paul et al., 2002, Nature Biotechnology, 19, 505; Miyagishi and Taira, 2002, Nature Biotechnology, 19, 497; Lee et al., 2002, Nature Biotechnology, 19, 500; and Novina et al., 2002, Nature Medicine. An expression vector may encode one or both strands of a nucleic acid duplex, or a single self-complementary strand that self hybridizes into a nucleic acid duplex. The nucleic acid sequences encoding nucleic acid molecules can be operably linked to a transcriptional regulatory element that results expression of the nucleic acid molecule in the target cell. Transcriptional regulatory elements may include one or more transcription initiation regions (e.g., eukaryotic pol I, II or III initiation region) and/or transcription termination regions (e.g., eukaryotic pol I, II or III termination region). The vector can optionally include an open reading frame (ORF) for a protein operably linked on the 5′ side or the 3′-side of the sequence encoding the nucleic acid molecule; and/or an intron (intervening sequences).

The nucleic acid molecules or the vector construct can be introduced into the cell using suitable formulations. One preferable formulation is with a lipid formulation such as in Lipofectamine™ 2000 (Invitrogen, CA, USA), vitamin A coupled liposomes (Sato et al. Nat Biotechnol 2008; 26:431-442, PCT Patent Publication No. WO 2006/068232). Lipid formulations can also be administered to animals such as by intravenous, intramuscular, or intraperitoneal injection, or orally or by inhalation or other methods as are known in the art. When the formulation is suitable for administration into animals such as mammals and more specifically humans, the formulation is also pharmaceutically acceptable. Pharmaceutically acceptable formulations for administering oligonucleotides are known and can be used. In some instances, it may be preferable to formulate dsRNA in a buffer or saline solution and directly inject the formulated dsRNA into cells, as in studies with oocytes. The direct injection of dsRNA duplexes may also be done. Suitable methods of introducing dsRNA are provided, for example, in U.S. 2004/0203145 and U.S. 20070265220.

Polymeric nanocapsules or microcapsules facilitate transport and release of the encapsulated or bound dsRNA into the cell. They include polymeric and monomeric materials, especially including polybutylcyanoacrylate. The polymeric materials which are formed from monomeric and/or oligomeric precursors in the polymerization/nanoparticle generation step, are per se known from the prior art, as are the molecular weights and molecular weight distribution of the polymeric material which a person skilled in the field of manufacturing nanoparticles may suitably select in accordance with the usual skill.

Nucleic acid moles may be formulated as a microemulsion. A microemulsion is a system of water, oil and amphiphile which is a single optically isotropic and thermodynamically stable liquid solution. Typically microemulsions are prepared by first dispersing an oil in an aqueous surfactant solution and then adding a sufficient amount of a 4th component, generally an intermediate chain-length alcohol to form a transparent system. Surfactants that may be used in the preparation of microemulsions include, but are not limited to, ionic surfactants, non-ionic surfactants, Brij 96, polyoxyethylene oleyl ethers, polyglycerol fatty acid esters, tetraglycerol monolaurate (ML310), tetraglycerol monooleate (MO310), hexaglycerol monooleate (PO310), hexaglycerol pentaoleate (PO500), decaglycerol monocaprate (MCA750), decaglycerol monooleate (MO750), decaglycerol sequioleate (SO750), decaglycerol decaoleate (DA0750), alone or in combination with cosurfactants. The cosurfactant, usually a short-chain alcohol such as ethanol, 1-propanol, and 1-butanol, serves to increase the interfacial fluidity by penetrating into the surfactant film and consequently creating a disordered film because of the void space generated among surfactant molecules.

### EXAMPLES

The present methods, thus generally described, will be understood more readily by reference to the following examples, which are provided by way of illustration and are not intended to be limiting of the present methods and kits.

### Example 1

### Differentially Regulated lncRNAs in Melanoma Cells

One microgram of total RNA was labeled and hybridized to NCode human microarrays (Life Technologies™, Carlsbad, Calif., USA) and labeled according to the manufacturer's protocols (Life Technologies Corp., Carlsbad, Calif.). An Agilent 2 μm high resolution C scanner (Cat. # G2365CA) was used to scan the slides and the data was normalized and analyzed using GeneSpring software (Agilent Technologies). The NCode human array contains over 10,000 putative lncRNAs (>200 nt) including most of the known lncRNAs in human. Lack of coding potential was estimated by a previously described algorithm [11] that scores various characteristics of protein-coding genes, including open reading frame length, synonymous/non-synonymous base substitution rates and similarity to known protein. These arrays are the first generation of tools designed to investigate the dynamic expression of a large subset of lncRNAs in human to identify candidate genes for more detailed functional analysis. In addition to the lncRNA content, probes targeting mRNA content from RefSeq are also included, allowing discovery of coordinated expression with associated protein-coding genes.

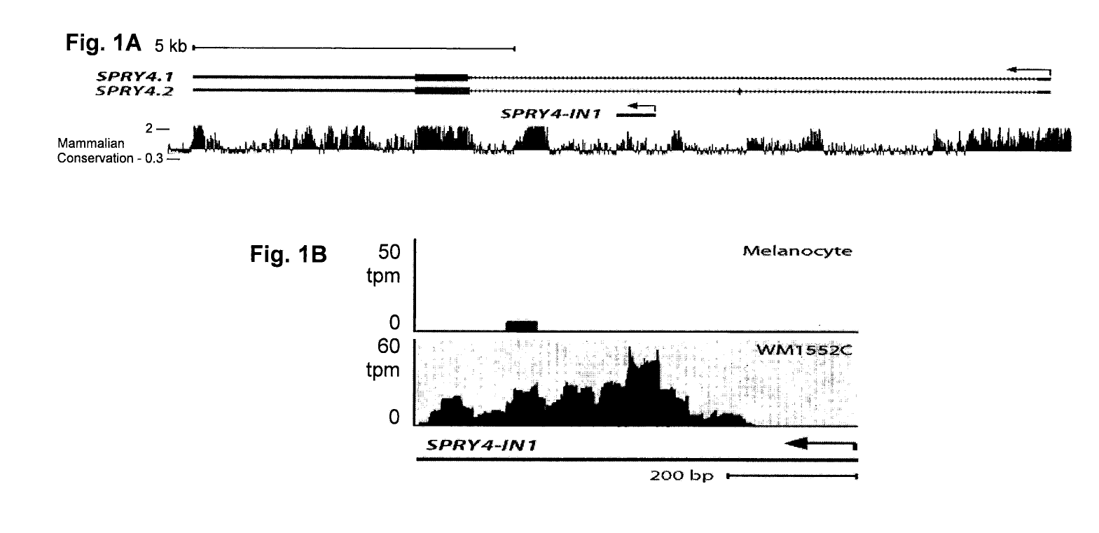

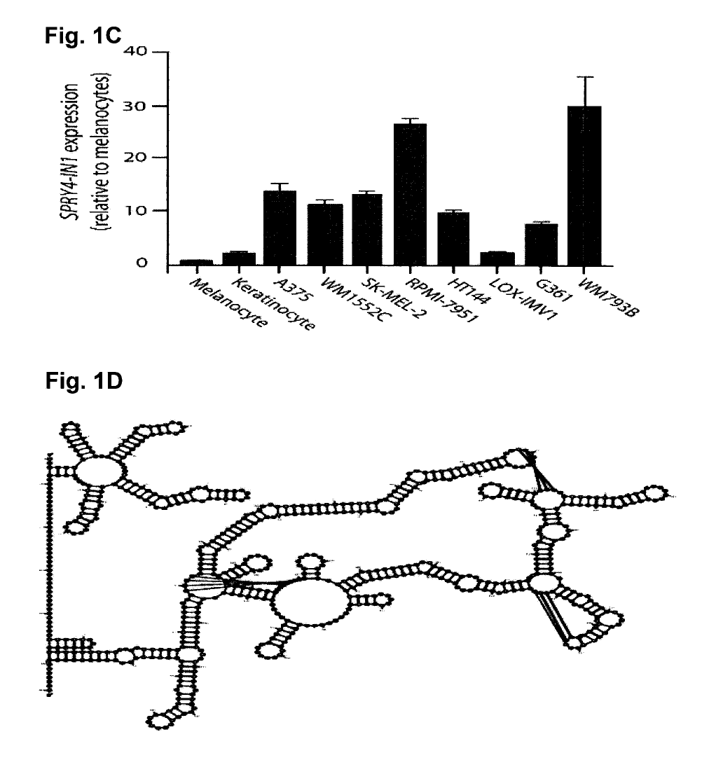

To identify lncRNAs involved in melanoma, total RNA from a stage III melanoma cell line (WM1552C), melanocytes, and keratinocytes, was analyzed using a non-coding RNA microarray (Ncode human array from Life Technologies). NCode human microarrays contain probes to target 12,784 lncRNAs and 25,409 mRNAs. In total, we identified 77 lncRNAs that were significantly differentially expressed (P<0.015; fold-change) in WM1552C relative to melanocytes. In addition to cell line profiling, 29 independent melanoma patient samples (graded as primary in situ, regional metastatic, distant metastatic and nodal metastatic), and six normal skin samples were also analyzed using the same microarrays. The differential lncRNA expression is presented as a hierarchical cluster (FIG. 6). Hierarchical clustering was done using the GeneSpring™ software (Agilent Technologies) and R package. The primary criteria in candidate selection for functional studies was whether the differentially expressed lncRNAs in melanoma cell lines were also differentially expressed in patient samples. Four candidate non-coding RNAs were screened initially (FIGS. 1A,B and 7A-D). lncRNA SPRY4-IT1 (Genbank Accession ID AK024556; SEQ ID NO: 1) is one such candidate that differentially expressed in both melanoma cell lines and patients samples relative to melanocytes.

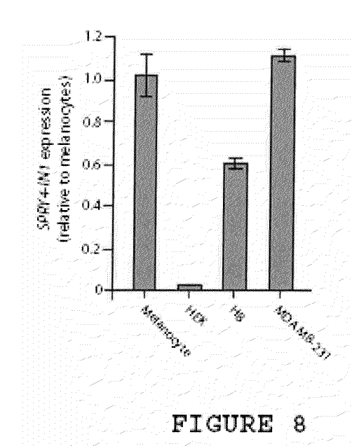

SPRY4-IT1 was selected for functional studies based on the criteria above. SPRY4-IT1 expression was further confirmed by deep-sequencing. SPRY4-IT1 expression was more than 12-fold higher in melanoma cells (WM1552C) relative to melanocytes. A comparison of SPRY4-IT1 in kidney, blood, and breast cell lines revealed expression to be equal to that of melanocytes or less (FIG. 8). We then measured the expression levels of SPRY4-IT1 (FIG. 1C) as well as the SPRY4 ORF (FIG. 9) in seven additional non-pigmented melanoma cell lines (WM793B, A375, SKMEL-2, RPMI 7951, HT-144, LOX-IMV1, and G361) by qRT-PCR and the results showed that the expression of both was elevated in most of the melanoma cell lines relative to control melanocytes.

### Example 2

### Structural Prediction of SPRY4-IT1

The most recent versions of RNAfold and RNAstructure were employed for generating RNA secondary structures. The partition function algorithm was chosen for two reasons: (i) it produces a structure almost identical to the minimum free energy algorithm with RNAfold with few proximal sub-optimal structures, and (ii) it is required for subsequent prediction of pseudoknots with ProbKnot (included in RNAstructure).

The evolutionary conservation of secondary structures was conducted with the consensus-based programs RNAz and SISSIz on the Enredo-Pecan-Ortheus 31-way eutherian mammal genome alignment from ENSEMBL. Orthologous sequences to SPRY4-IT1 were selected and realigned with MAFFT using the mafft-ginsi algorithm. Sliding window ranges of 100 nt window with 25 nt slide, 150 nt window with 50 nt slide, and 300 nt window with 100 nt slide were tested with both RNAz and SISSIz, using parameters “-d” and “-d-t-n 200-p 0.02”, respectively.

SPRY4-IT1 is a 687 nt unspliced, polyadenylated transcript originally identified in adipose tissue and is transcribed from the intronic region of the SPRY4 gene (FIG. 1A). This region is not conserved beyond the primate genomes and there is no EST expression detected in mouse. To determine whether the SPRY4-IT1 RNA contained any particular secondary structural features, the SPRY4-IT1 genomic sequence was submitted to secondary structure and pseudoknot prediction using two different programs that implement an RNA partition function algorithm. The results appear in FIG. 1D, wherein blue lines indicate positions of pseudo knots, and red base-pairing indicates regions of consensus structure between the two algorithms. Several helical regions are common to both algorithms, including a large stem-loop from positions 220 to 321 (FIG. 1D). The latter encompasses one of two non-repeat associated “pyknons”, putative regulatory motifs that are non-randomly distributed throughout the genome. In addition, three putative pseudoknots (i.e. nested helices) are predicted by ProbKnot, which boasts high sensitivity and positive prediction value. No compatible structures appear to be significantly conserved throughout a multiple alignment of orthologous sequences from 31 eutherian mammals. The likelihood that it could fold into long stable hairpin structures (FIG. 1B), suggests that SPRY4-IT1 may function intrinsically as a RNA molecule.

### Example 3

### Expression Profiling of SPRY4 and SPRY4-IT1 in Human Tissue

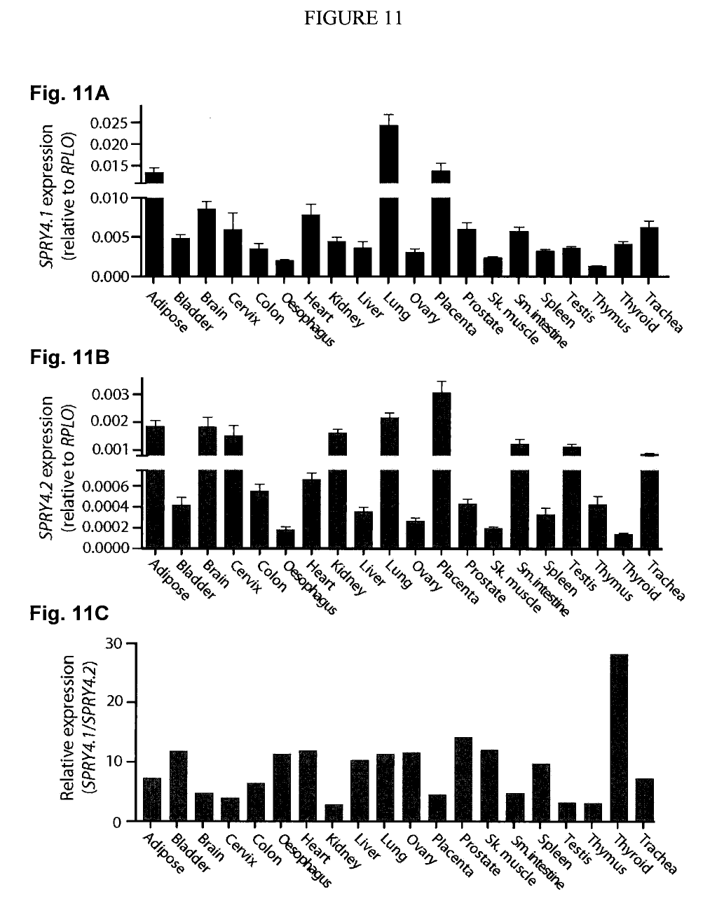

SPRY4 is an inhibitor of the receptor-transduced mitogen-activated protein kinase (MAPK) signaling pathway. It functions upstream of RAS activation and impairs the formation of active GTP-RAS. SPRY4 is down-regulated in non-small cell lung cancer and inhibits cell growth, migration, and invasion in transfected cell lines, suggesting it may function as a tumor suppressor. SPRY4 occurs in two alternately spliced isoforms, termed SPRY4.1 and SPRY4.2 (FIG. 1A), the latter of which retains an additional exon that results in translation initiating from an alternate start codon. To better understand where SPRY4 functions and the relative expression of the two isoforms, qRT-PCR was used to measure the expression of SPRY4.1 and SPRY4.2 across 20 human tissues (FIG. 11). The results showed that both isoforms are expressed in all tissues examined, with the highest expression found in the lung and placenta and lowest in the thymus and oesophagus. SPRY4.1 was found to be the more abundant isoform, occurring in diverse ratios (relative to SPRY4.2) across different tissues, ranging from 2.7:1 in kidney to 28:1 in thyroid. Despite the differences in abundance, the expression profiles of SPRY4.1 and SPRY4.2 were positively correlated (R=0.75; Pearson correlation).

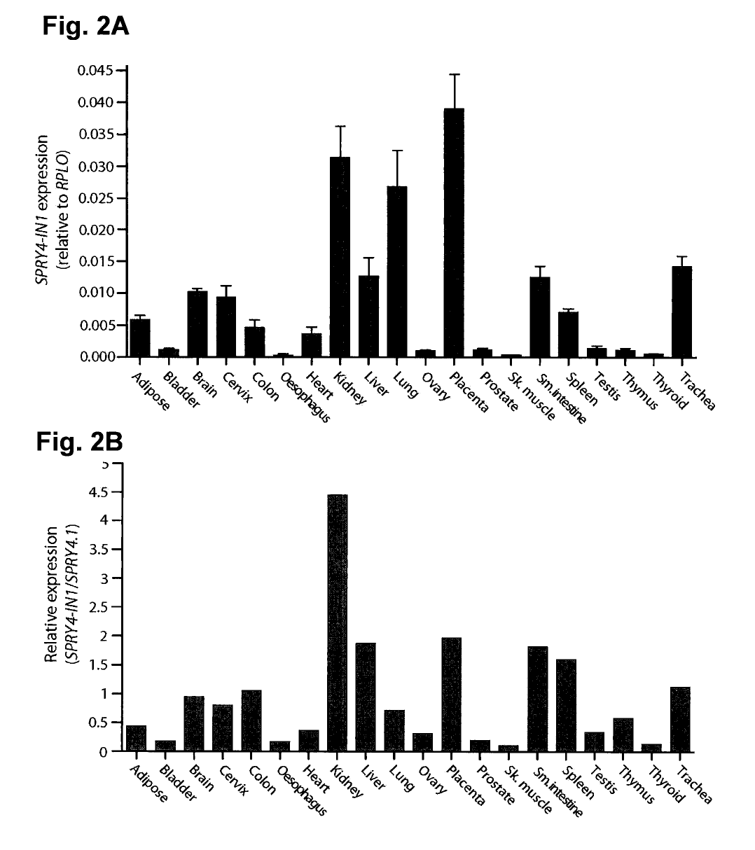

Given the intronic position of SPRY4-IT1 within SPRY4, it was next determined whether the expression of SPRY4-IT1 and SPRY4 were linked. Therefore, in order to ascertain any linkage the relative expression of SPRY4-IT1 across the same panel of 20 human tissues was examined (FIG. 2A). Interestingly, in several tissues, SPRY4-IT1 was more highly expressed than SPRY4.1, occurring at ratios as high as 4.5:1 in kidney (FIG. 2B). Furthermore, the range in expression for SPRY4-IT1 across the 20 different tissues was much greater than that of SPRY4; SPRY4-IT1 varied by as much as 111-fold (placenta vs oesophagus) compared to SPRY4.1 which varied by a maximum of ˜10-fold (thyroid vs kidney). Despite the variation in abundance and range, the expression profile of SPRY4-IT1 was correlated with both SPRY4.1 (R=0.62; Pearson correlation) and SPRY4.2 (R=0.84; Pearson correlation). The similar expression profiles between SPRY4-IT1 and SPRY4 suggests that SPRY4-IT1 and SPRY4 may share the same transcriptional regulatory factors or indeed may be processed directly from the intron of SPRY4. In the latter scenario, the higher abundance of SPRY4-IT1 could be explained by higher stability of the lncRNA relative to the mRNA.

Total RNA was isolated using Trizol (Life Technologies) with subsequent quantification by using an Agilent 2100 Bioanalyzer (Agilent Technologies, Santa Clara. Calif., USA). 1 μg of total RNA was reverse transcribed using the High Capacity cDNA kit (Applied Biosystems Inc., Foster City, Calif., USA), and qRT-PCR was carried out using TaqMan Assays in the 7500 Real-Time PCR System (Applied Biosystems) according to the manufacturer's protocols. SDS1.2.3 software (Applied Biosystems) was used for comparative Ct analysis with GAPDH serving as the endogenous control.

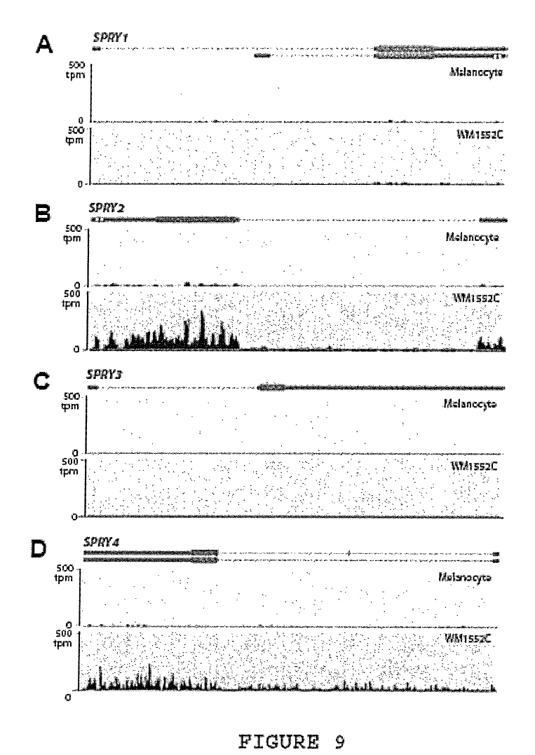

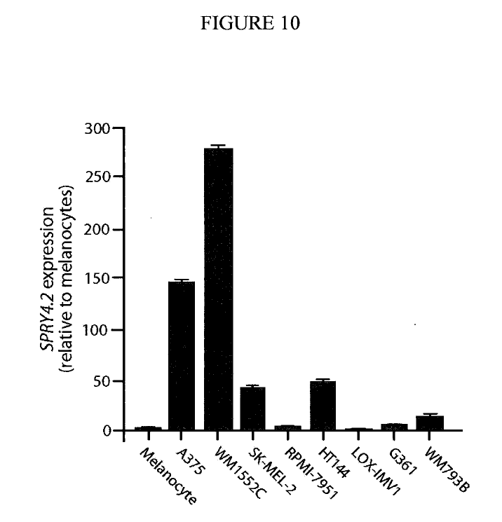

Next generation sequencing experiments show that SPRY1 and SPRY3 have little or no expression in either melanoma or melanocytes, but both SPRY2 and SPRY4 are highly expressed in melanoma cells compared to melanocytes (FIG. 10).

For the human tissue expression analysis, total RNA from 20 different tissues was purchased from Ambion. 1 ug was oligo-dT reverse transcribed using Superscript III (Life Technologies) and qRT-PCR was carried out using the TaqMan Noncoding RNA Assays (SPRY4-IT1) and TaqMan Gene Expression Assays (SPRY4.1 and SPRY4.2) in the 7900 Real-Time PCR System (Applied Biosystems) according to the manufacturer's protocols. SDS2.3 software (Applied Biosystems) was used for comparative Ct analysis with RPLO serving as the endogenous control.

### Example 4

### SPRY4-IT1 and SPRY4 Expression in Patient Tissue Samples

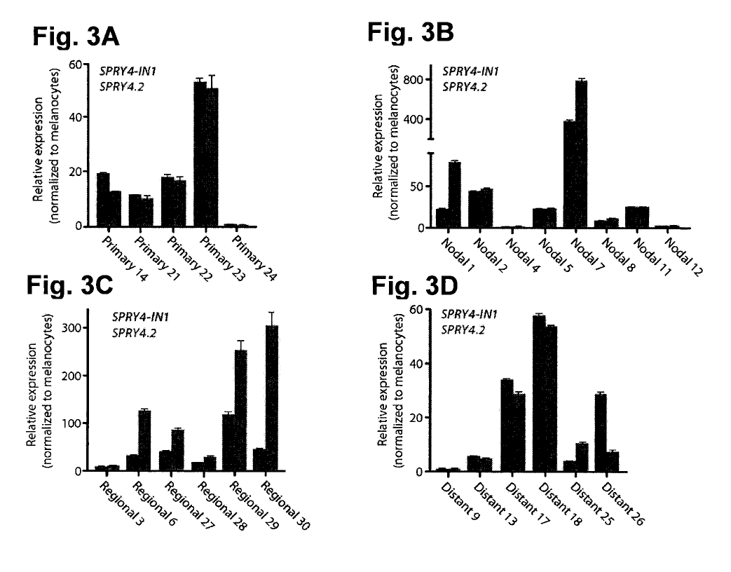

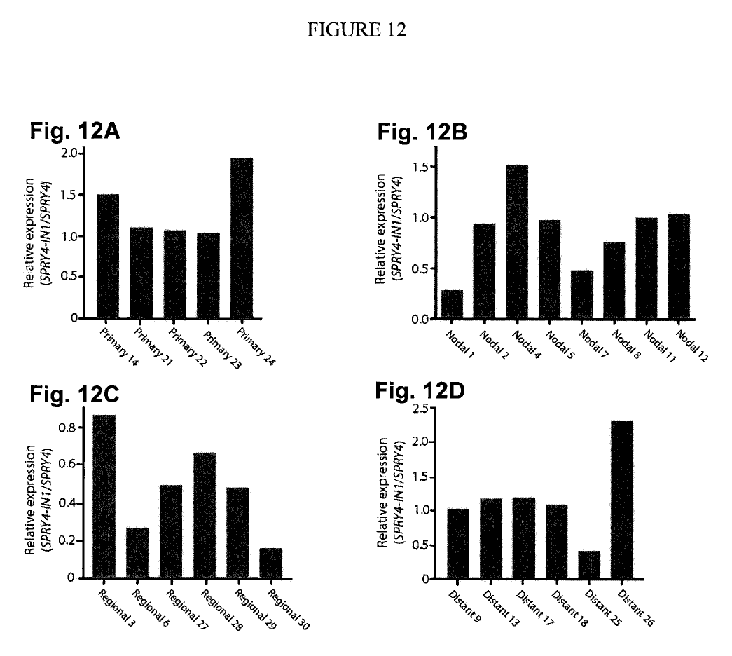

The expression of SPRY4-IT1 and SPRY4 in 25 melanoma patient samples was then examined using quantitative RT-PCR (FIGS. 3A-D). The expression of both SPRY4-IT1 and SPRY4.2 varied considerably between patient samples but their relative expression levels were highly correlated (R=0.95; FIG. 12). These results validated the microarray expression data, showing that SPRY4-IT1 was up-regulated in melanoma patient samples compared to the melanocyte control (FIG. 3A-D).

Additionally, relative expression of SPRY4-IT1 to SPRY4.2 in primary, nodal metastasis, regional metastasis, and distant metastasis in melanoma patient samples. The results are shown in FIG. 12.

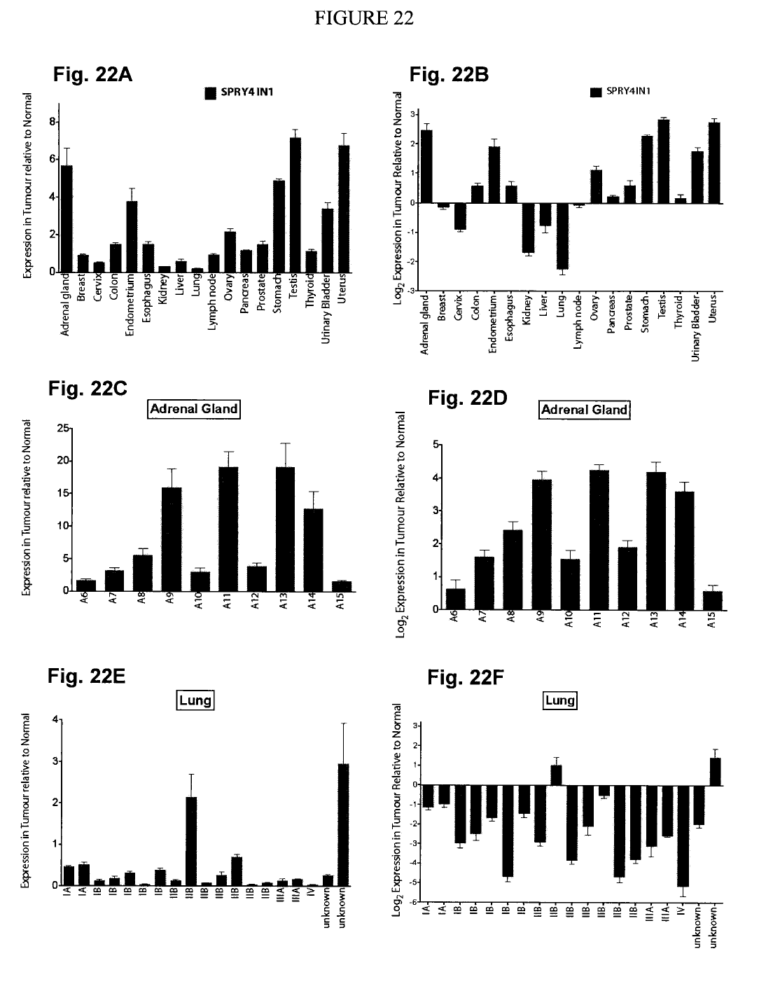

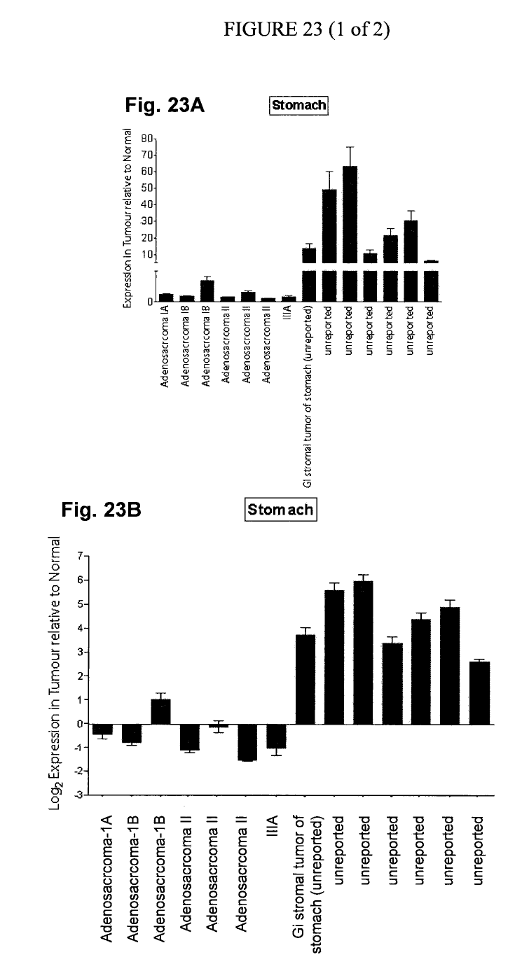

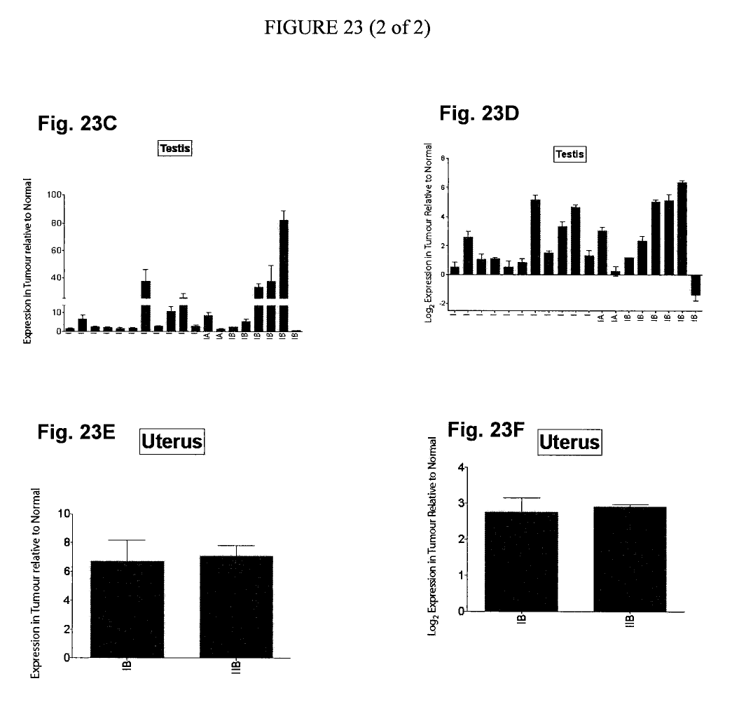

Finally, the expression of SPRY4-IT1 in tumor cells of 18 organs was measured and compared to normal tissue expression. The results, which are shown in FIGS. 22A and B, show that the highest level of expression was found in the adrenal gland, the stomach, the uterus, and the testis. A number of samples from each of the organs with highest expression levels of SPRY4-IT1 were then subjected to RT-PCR to calculate the expression level of SPRY4-IT1 in each sample. The results are given in FIG. 22C-FIG. 23, and confirm the presence of ectopic SPRY4-IT1 in tumor cells other than melanoma.

### Example 5

### RNAi to Knock-Down SPRY4-IT1 in Melanoma Cells

Five different Stealth RNAi™ siRNAs that targeted SPRY4-IT1 RNA and a Scramble Stealth RNAi™ siRNA control were used to knock down SPRY4-IT1 RNA in melanoma cells (Life Technologies). The Stealth RNAi™ siRNA molecules are 25 base-pair double-stranded RNA oligonucleotides with proprietary chemical modifications. The BLOCK-iT RNAi designer was used to find gene-specific 25 nucleotide Stealth RNAi™ siRNA molecules. It uses gene-specific targets for RNAi analysis and reports up to 10 top scoring Stealth RNAi™ siRNA targets. The freeze-dried siRNAs were dissolved in RNase free-water and stored as aliquots at −20° C. The siRNA with the sequence GCTTTCTGATTCCAAGGCCTATTAA (SEQ ID NO: 2) yielded the highest degree of SPRY4-IT1-knockdown.

### Cell Culture Conditions and Transfection

Transfection was done with Lipofectamine™ RNAiMax (Life Technologies) in 6 well plates, 6, 12 and 18 nM RNAi duplexes were diluted in 500 μL serum free medium, mixed gently and 5 μL of Lipofectamine™ RNAiMAX was added to each well containing the diluted RNAi molecules. This mixture was incubated for 20 minutes at room temperature before the transfection. 250,000 cells were diluted in complete Tu growth medium (without antibiotics) and plated in each well. RNAi duplex-Lipofectamine™ RNAiMAX complexes were added to each well and mixed gently by rocking the plate. Cells were incubated for 48 hours at 37° C. in a CO₂ incubator and gene knockdown was assessed by qRT-PCR.

### Northern Blot Analysis

Total RNA concentrated from each sample (20 μg) was separated in 15% TBE-urea polyacrylamide gels by electrophoresis. The RNA was electroblotted onto nylon membranes, cross-linked by ultraviolet light, prehybridized in Ultrahyb-Oligo (Ambion) for 30 min at 42° C., and hybridized at 100 nM with a 5′-biotinylated anti-miRNA DNA oligonucleotide (TCCACTGGGCATATTCTAAAA; SEQ ID NO: 3) at 42° C. overnight. The blots were then washed, and the signal was detected by chemiluminiscence (Brightstar Detection kit, Ambion). Anti-U6 probes (10 pM) were used as a reference control.

### RNA-FISH Analysis

Locked nucleic acid (LNA)-modified probes for human lncRNA SPRY4-IT1 (5′-FAM-TCCACTGGGCATATTCTAAAA-3′-FAM; SEQ ID NO: 3) and a negative/Scramble control (5′-TYE665-GTGTAACACGTCTATACGCCCA-3′-TYE665 (SEQ ID NO: 4), miRCURY-LNA detection probe, Exiqon) were used for RNA in situ hybridization. In situ hybridization was performed using the RiboMap in situ hybridization kit (Ventana Medical Systems Inc) on a Ventana machine. The cell suspension diluted to 10,000 cells per 100 μL was pipetted into clonal rings on the autoclaved glass slides. The following day, the clonal rings were removed; slides were washed in PBS and fixed in 4% paraformaldehyde and 5% acetic acid. After acid treatment using hydrochloride-based RiboClear reagent (Ventana Medical Systems) for 10 min at 37° C., the slides were treated with the ready-to-use protease 3 reagent. The cells were hybridized with the antisense LNA riboprobe (40 nM) using RiboHybe hybridization buffer (Ventana Medical Systems) for 2 h at 58° C. after an initial denaturing prehybridization step for 4 min at 80° C. Next, the slides were subjected to a low-stringency wash with 0.1×SSC (Ventana Medical Systems) for 4 min at 60° C., and then two further washing steps with 1×SSC for 4 min at 60° C. These slides were fixed in RiboFix and counterstained with 4′-6′ diamidino-2-phenylindole (DAPI), in an antifade reagent (Ventana). The images were acquired using a Nikon A1R VAAS laser point- and resonant-scanning confocal microscope equipped with a single photon Ar-ion laser at 60× with 4× zoom.

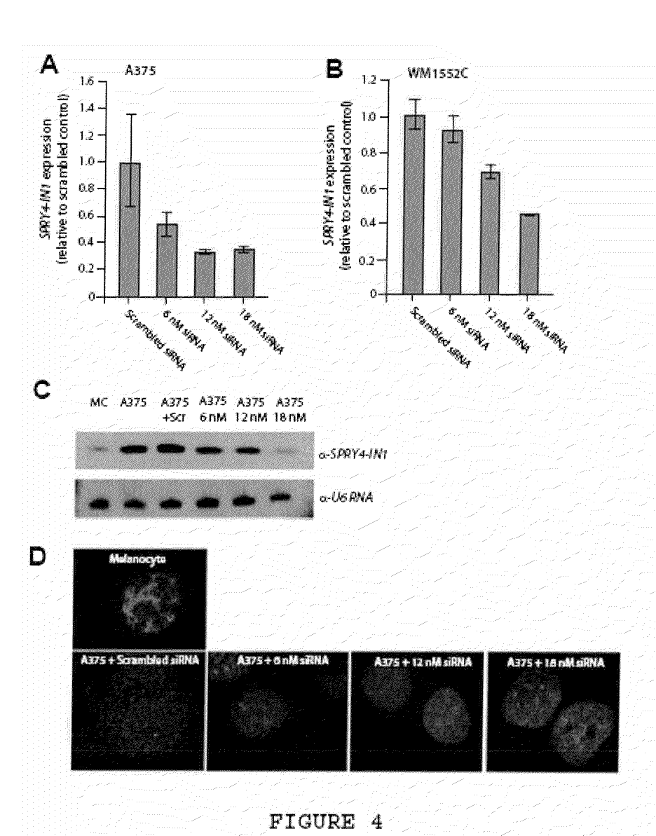

To probe the functional role of SPRY4-IT1, Stealth RNAi was used to down-regulate SPRY4-IT1 expression in melanoma cells. Five different stealth RNAi molecules were tested for their knockdown efficiency, the most efficient of which (stealth RNAi 594) was selected for subsequent biological studies. To determine the optimal concentration for knockdown, several different concentrations of stealth siRNA were examined in the melanoma cell lines A375 and WM1552C (FIGS. 4A & 4B). When these cells were transfected with 6 nM of stealth siRNA, it showed a 45% SPRY-1-IT1 silencing in A375 cells, but no significant changes were observed in WM1552C cells. However, 18 nM of stealth RNAi yielded at least 60% knock-down in both cell lines (WM1552C and A375). These results were validated by northern blot analysis (FIG. 4C). Though a high level of SPRY4-IT1 knock-down occurred with 30 nM siRNA, significant cell death also occurred. Therefore, subsequent cell biology studies were performed with a maximum of 18 nM stealth siRNA. Stealth RNAi-transfected A375 cells were also screened for their expression of SPRY4, revealing no changes in expression (indicating that the down-regulation of the lncRNA SPRY4-IT1 did not effect the expression of the SPRY4 ORF) (FIG. 10).

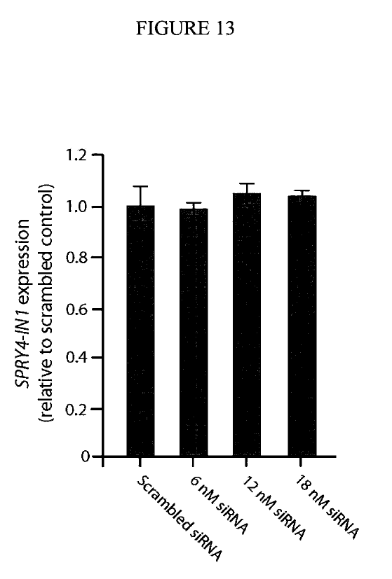

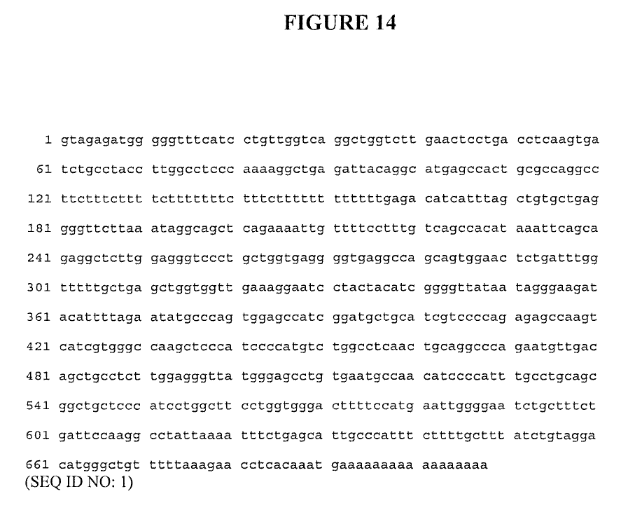

Given the correlated expression of SPRY4-IT1 and SPRY4, the effect of SPRY4-IT1 knockdown on SPRY4 was investigated in A375 cells using qRT-PCR. It was found that the level of SPRY4 expression was not significantly altered following SPRY4-IT1 knockdown relative to the scrambled siRNA control (FIG. 13). This confirms that the RNAi knockdown strategy does not appreciably alter the expression levels of the host SPRY4 transcript. The phenotypic effects observed following knockdown of SPRY4-IT1 result directed from SPRY4-IT1 and not from the regulation of SPRY4 by SPRY4-IT1.

The expression of lncRNA SPRY4-IT1 in A375 cell lines and melanocytes was then examined by in situ hybridization using a locked nucleic acid (LNA) FAM-labeled probe (see Methods). RNA FISH showed that SPRY4-IT1 is localized as a punctate pattern in the nucleus, but the majority of the signal was observed in the cell cytoplasm (FIG. 4D). Consistent with previous qRT-PCR results (FIG. 1C), RNA FISH also revealed that SPRY4-IT1 was highly expressed in A375 melanoma cell lines compared to melanocytes. The dose-dependent reduction of RNA-FISH signal in A375 cells transfected with different concentrations of SPRY4-IT1-targeted siRNAs show that the probe was specifically detecting the SPRY-1-IT1 transcript.

### Example 6

### SPRY4-IT1 Inhibition Effects Metabolic Viability and Cell Death

### Metabolic Viability by MTT Assay

To investigate the possible role of SPRY4-IT1 on the growth of melanoma cells, the metabolic viability was assessed using a colorimetric assay, which involves the conversion of MTT in active mitochondria of living cells to formazan. The amount of formazan correlates with the number of viable cells. MTT (3-(4,5-dimethyl-2-yl)-2,5-diphenyl-2H-tetrazolium bromide) was purchased from Roche. Cells were plated in 96 well plates (5000 cells/100 μL/well). After 48 h of transfection, 20 μL MTT solution was added and the cells were incubated at 37° C. in the dark for 4 h. The generated formazan OD was measured at 490 nm to determine the cell viability on the Flex station (Molecular Devices).

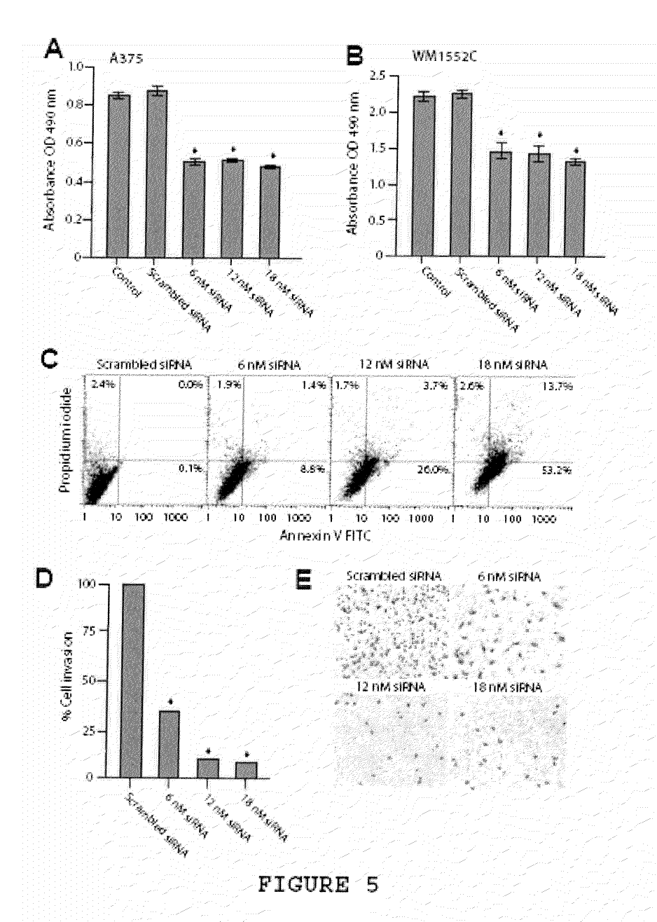

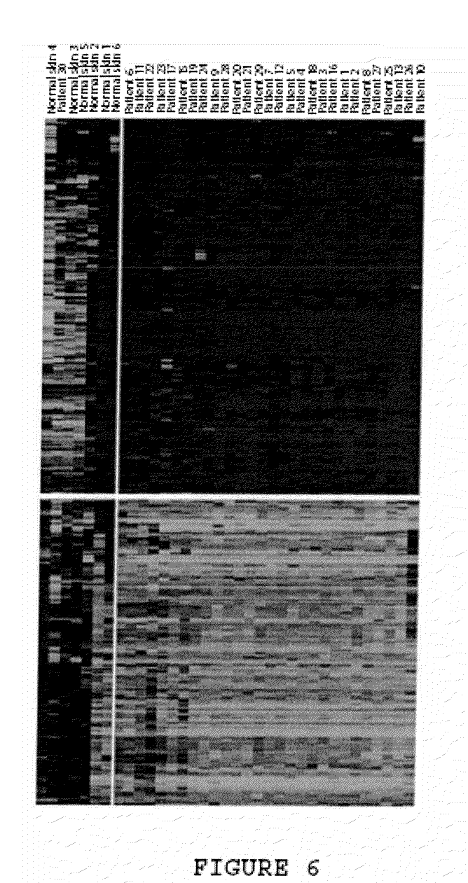

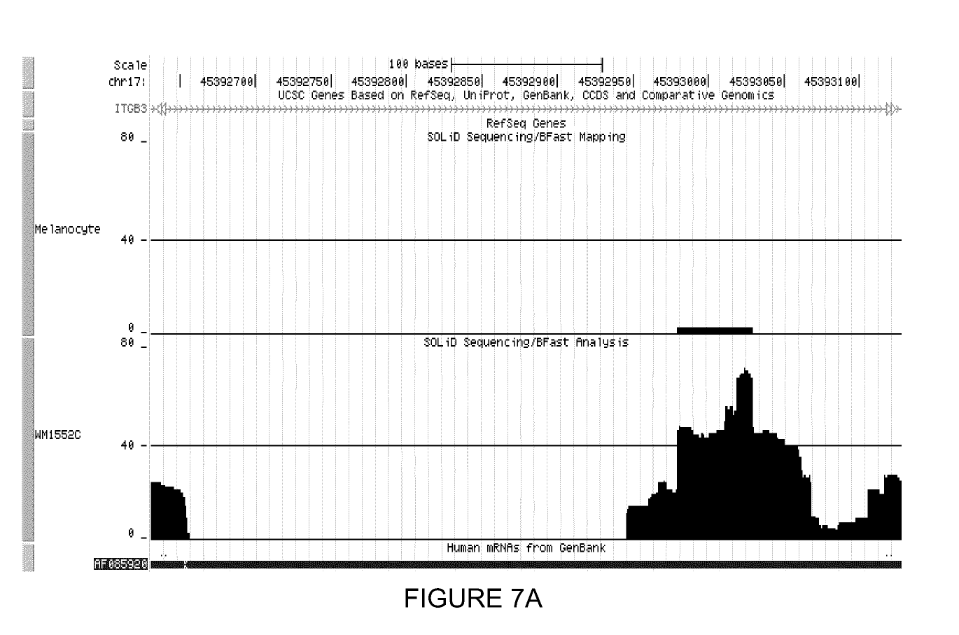

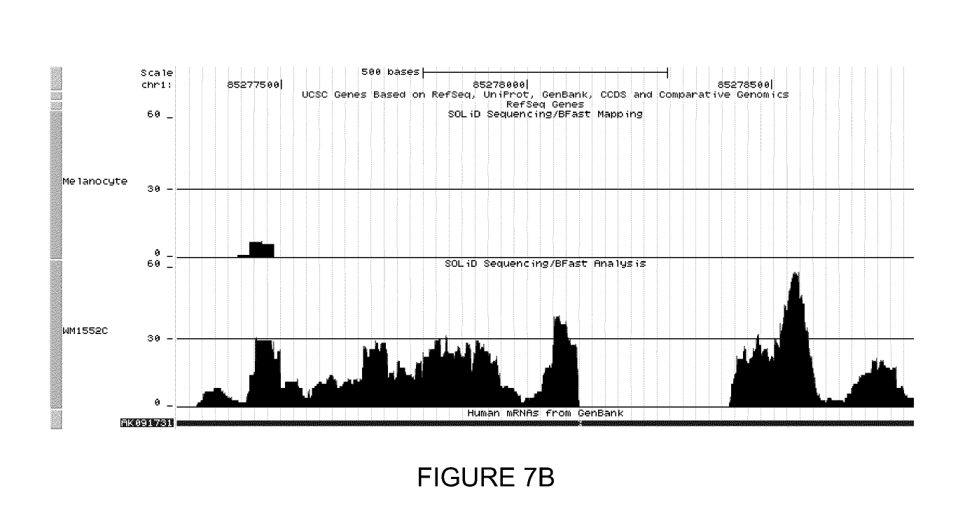

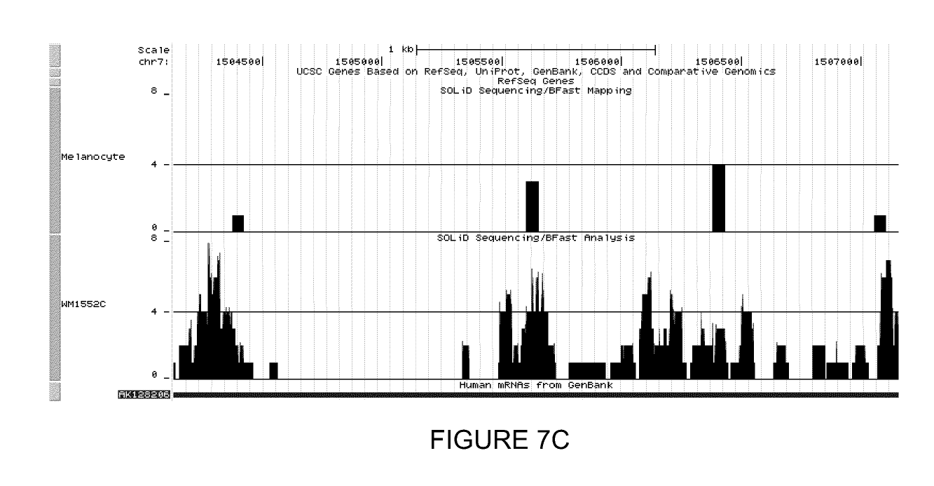

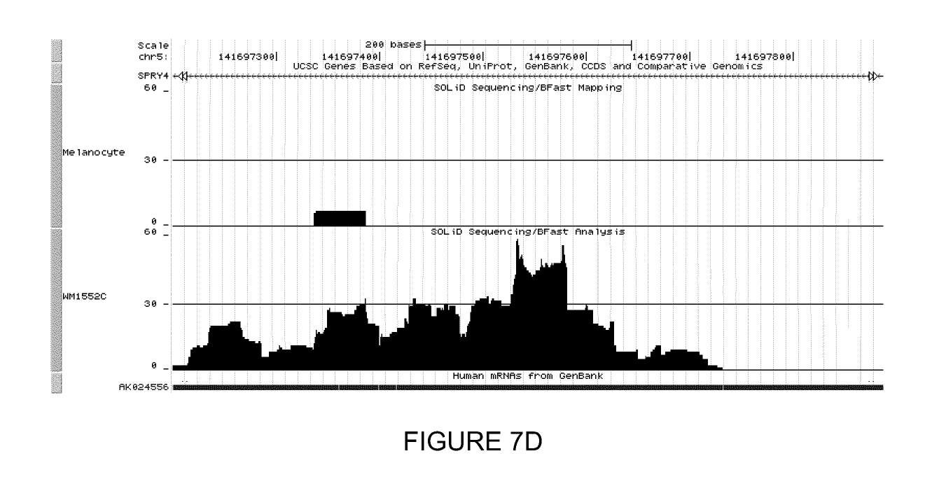

A375 melanoma cells knocked-down with Stealth siRNA showed a 50% decrease in metabolic viability 48 hours after transfection, whereas WM1552C cells showed a 30% decrease in viability (FIGS. 5A & 5B). The MTT assay demonstrates that the down-regulation of SPRY4-IT1 expression decreases cell growth in melanoma cells.

### Phosphatidylserine Externalization

Next, the effects of SPRY4-IT1 knock-down on apoptosis were investigated. Apoptosis was detected by labeling phosphotidylserine using FITC-conjugated Annexin V in unfixed cells. Cell death was studied by flow cytometry using Annexin V. Annexin V binds to the negatively charged phospholipids located on the inner surface of the plasma membrane. Annexin V conjugated to fluorescein together with propidium iodide is used to detect non-apoptotic live cells (Annexin V negative, PI negative), early apoptotic cells (Annexin V positive, PI negative) and late apoptotic or necrotic cells (PI positive). Transfected (stealth siRNA) and untransfected cells were washed twice with PBS, trypsinized and washed again with PBS. Cells were re-suspended in binding buffer (10 mM HEPES+10 mM NaOH−pH 7.4, 140 mM NaCl, 2.5 mM CaCl₂) at a density of 0.5-1×10⁶ cell/mL. To the 100 μL of cell suspension, 3 μL of Annexin V FITC (B.D. Pharmingen) and 10 μL of PI (10 μg/mL) was added and gently vortexed. The cells were incubated at room temperature for 15 min in the dark. To each of the samples, 400 μL of binding buffer was added and placed on ice. Flow cytometric measurements were carried using a FACS caliber flow cytometer (Becton and Dickinson, USA). Green fluorescence due to Annexin V-FITC was collected on the FL1 channel and red fluorescence due to PI was collected on the FL2 channel on a log scale. A minimum of 10,000 cells per sample was acquired and analyzed using CcllQuest software (Becton and Dickinson).

The percentage of Annexin V positive-negative and PI positive-negative cells was estimated by eating the cell population. A 375 untreated or Scrambled stealth siRNA-treated cells showed minimum annexin positive cells 48 hours after transfection (FIG. 5C). The fraction of annexin positive cells with 6 nM of stealth siRNA was 9%. This was increased to 26% at 12 nM and 53% when 18 nM of Stealth siRNA used for transfection. Interestingly, no major differences were observed in propidium iodide positive cells indicating that the knockdown of SPRY4-IT1 induces cell death primarily through apoptosis, not necrosis. The effect of SPRY4-IT1 knockdown on the invasion of A375 melanoma cells was also examined (FIGS. 5D & 5E).

### Example 7

### SPRY4-IT1 Inhibition Effects Cell Invasion

### Invasion Assays

BD BioCoat™ growth factor reduced insert plates (Matrigel™ Invasion Chamber 12 well plates) were prepared by rehydrating the BD Matrigel™ matrix coating in the inserts with 0.5 mL of serum-free complete Tu media for 2 h at 37° C. The re-hydration solution was carefully removed from the inserts, 500 μL complete Tu (2% FBS) was added to the lower wells of the plate. 1×10⁴ transfected and untransfected cells suspended in 500 μL of serum-free complete Tu media was added to the top of each insert well. Invasion assay plates were incubated for 48 h at 37° C. Following incubation, the non-invading cells were removed by scrubbing the upper surface of the insert. The cells on the lower surface of the insert were stained with crystal violet and each trans-well membrane mounted on a microscope slide for visualization and analysis. The slides were scanned in Scanscope and the number of cells migrating was counted using Aperio software (Aperio Technologies). Data are expressed as the percent invasion through the membrane relative to the migration through the control membrane.

The results of the invasion assay demonstrate that knock-down of SPRY4-IT1 inhibits melanoma cell invasion by greater than 60% at 6 nM of Stealth siRNA and greater than 80% at 12 and 18 nM. This invasion defect is significant, even accounting for defects due to the loss of cell viability (>80% invasion defect at 12 and 18 nM Stealth siRNA with only a 50% loss of cell viability) (see FIGS. 5D and 5E).

### Example 8

### SPRY4-IT1-Induced Proliferation, Invasion, and Multinucleation of Melanocytes is Due to Modulation of Cancer-Related Target Genes

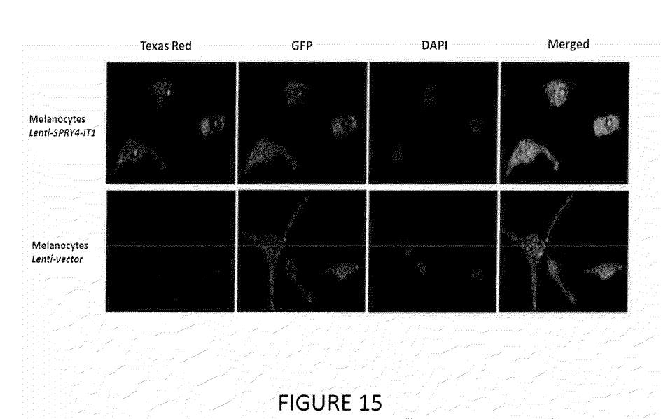

To confirm that modulation of cancer-related target genes such as DPP-IV, TNFRSF25, MCM2, CDK1, CDC20, XIAP, and Livin, results in the SPRY4-IT1-induced increase in cell proliferation, invasion, and multinucleation described in the examples above, RNA-FISH analysis was first performed as described in Example 5, supra, to detect expression of SPRY4-IT1 in cells infected with the lentiviral vector (control) and the lend-SPRY4-IT1 vector in melanocytes. The results are shown in FIG. 15, and show GFP expression as a control to indicate that the lentiviral vector has successfully incorporated into the genome. Intense nuclear foci indicate the presence of the longer (743 bp) version of the unprocessed SPRY4-IT1.

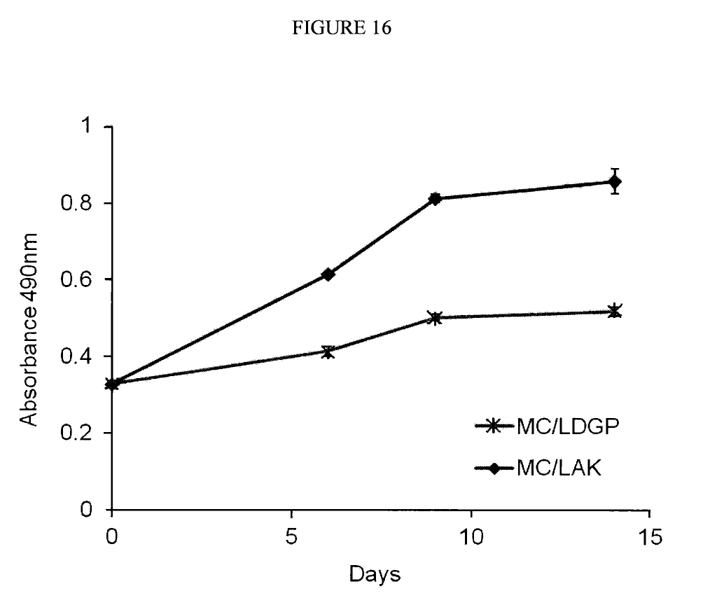

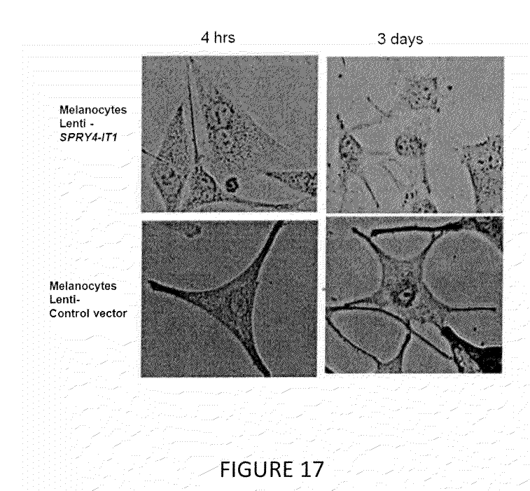

As shown in FIG. 16, ectopic expression of SPRY4-IT1 increases proliferation in the melanocytes engineered to ectopically express SPRY4-IT1 when compared to cells expressing empty vector. Further, the proliferating cells have been shown to increase in size and become multinucleated, as shown in FIG. 17.

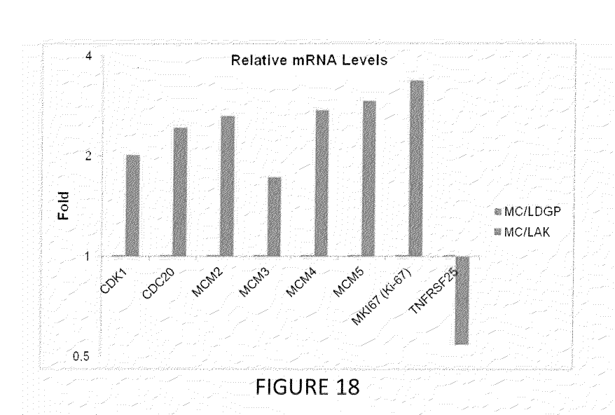

Using the methodology described above in Example 3, the RNA and protein content was analyzed using qRT-PCR and protein microarrays to identify the modulated genes. The proto-array data showed that expression of DPP-IV and Trail R2/DR5 were highly downregulated, and Hsp60, Hsp70, Livin, and XIAP were upregulated by enforced SPRY4-IT1 expression in melanocytes. DPP-IV was previously shown to be downregulated in human melanoma, and also suppresses IL-2 production and T-cell proliferation. In the qRT-PCR array, TNFRSF25 was confirmed as being downregulated, and Ki-67, CDK1, CDC20, MCM2, MCM3, MCM4, and MCM5 were highly upregulated, as shown in FIG. 18. Further, cell migration was shown in MC/LAK cells after four days in culture, but not by control MC/LDGP cells. Collectively, these data confirm the direct involvement of SPRY4-IT1 in melanoma development, and further confirm the a set of target genes implicated in cell proliferation and invasion.

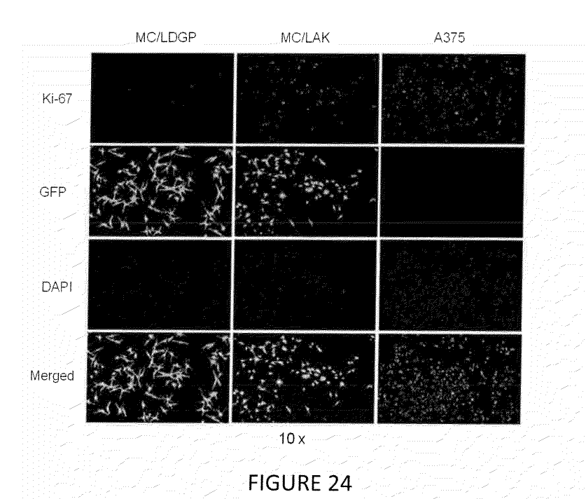

Staining of SPRY4-IT1-expressing MC/LAK cells and vector-only MC/LDGP cells revealed that only the SPRY4-IT1 cells expressed Ki-67, confirming the results from the qRT-PCR array as shown in FIG. 24, and consistent with the higher proliferation of SPRY4-IT1-expressing cells. FIG. 24 shows staining in melanocytes expressing SPRY4-IT1 as compared to cells expressing empty vector. Expression of Ki-67 is indicated in the top row. There is little or no expression of Ki-67 in MC/LDGP control cells, but high expression in MC/LAK cells and in the melanoma cell line A375, which was used as a positive control. This confirms the qRT-PCR results shown in FIG. 18.

In order to still further confirm that manipulation of target genes may reverse the melanoma-like phenotype observed in MC/LAK cells, SPRY4-IT1-expressing melanocytes are modified using shRNA to create knockdowns of MCM2, CDK1, CDC20, XIAP, and Livin. All lenti-shRNA premade constructs are purchased from Open-Biosystems. The final lentiviral packaging and cell line production is completed at the functional genomics core laboratory at Sanford Burnham Medical Research Institute. Except DPP-IV and TNERSF25, all genes will be knocked down with lentiviral shRNA. Since these two genes are downregulated in human melanoma, DPP-IV and TNFRSF25 constructs are separately synthesized to over-express these genes in MC/LAK cells. The SPRY4-IT1-expressing melanocyte cell lines engineered to over- and under-express the target genes are subjected to several assays:

First, the invasiveness and migration of transfected melanocytes are assayed by a modified form of the standard Boyden chamber assay (described by Kleinman, H. K., and Jacob, K., Invasion Assays, Curr. Protoc. Cell Biol., 2001), in which cell invasion by MC/LAK cells is compared to vector-only cells, MC/LDGP after culturing for four days, the long culture period taking into account the slow growth rate of melanocytes compared to melanoma cells.

In addition to standard invasion assays, Q3DM high throughput microscopy and invasion assay (HTM-IA) is also performed to quantify cell invasion. This technique, developed by Vala Scientific Corporation automated cell imaging team, allows for direct visualization and quantification of cell invasiveness, MTT and Brdu incorporation assays (described in Mosmann, T., Rapid calorimetric assay for cellular growth and survival: application to proliferation and cytoxicity assays, J. Immunol. Methods, 1983, 65(1-2), 55-63) (MTT Cell Proliferation Kit Roche) are then performed to assess proliferation and viability of target modulated SPRY4-IT1-expressing cells. Colony formation is measured in vitro by soft agar assays.

Further, an in vitro wound healing assay is performed to assess cell migration. First, the cells seeded on MatTek 1.5 mm tissue culture dishes and incubated until 90-95% confluent. The cell monolayers are scratched with a pipette tip across the entire diameter of the dish, and the dishes rinsed extensively with media to remove all cellular debris. The surface area is quantified immediately after wounding, and again at 20-minute intervals for up to 24 hours, using a Nikon Bio Station inverted microscope. The extent of wound closure is determined by calculating the ratio of the surface area between the remaining wound edges for each time point to the surface area of the initial wound. The data are presented as the percentage of wound closure relative to the control conditions for each experiment. The surface area is calculated using NIS Elements software, and each assay is performed in triplicate.

To confirm that SPRY-IT1 and its target genes induce apoptosis, cells are assayed using the standard Terminal dUTP Nicked-End Labeling (TUNEL) assay, as described in Gavrieli et al., Identification of programmed cell death in situ via specific labeling of nuclear DNA fragmentation, J. Cell Biol., 1992, 119(3), 493-501. To examine necrosis, membrane permeation is measured by the exclusion of Trypan Blue. Caspase 3/7 activity is also used to determine apoptosis. DEV-Dase Caspase 3/7 activity is detected using the caspase Glo 3/7 Assay kit (Promega). The Guava cell cycle assay is used to measure the distribution of cells in the G0/G1, S, and G2/M phases of the cell cycle, which identifies an effect of SPRY4-IT1 and its target gene expression on melanocyte cell division. The assay uses propidiumiodide (PI) to stain S phase DNA, which results in increased fluorescence intensity. For the controls, melanocytes carrying empty vector are used.

The collective results of these assays demonstrate that not only is a set of SPRY4-IT1 targets responsible for proliferation, invasion, and multinucleation, but, importantly, that manipulation of these same target genes may reverse this melanoma-like phenotype observed in MC/LAK cells

### Example 9

### SPRY4-IT1 Functions Through Interactions with Protein/RNA Partners

To confirm that SPRY4-IT1 functions through interactions with proteins and/or RNA partners, the SPRY4-IT1 regulatory region is first characterized to identify its transcriptional regulation and the molecular mechanism of SPRY4-IT1 processing, RNA decay and trafficking.

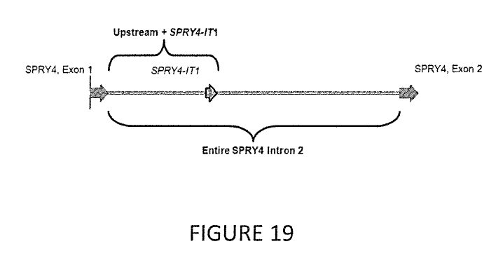

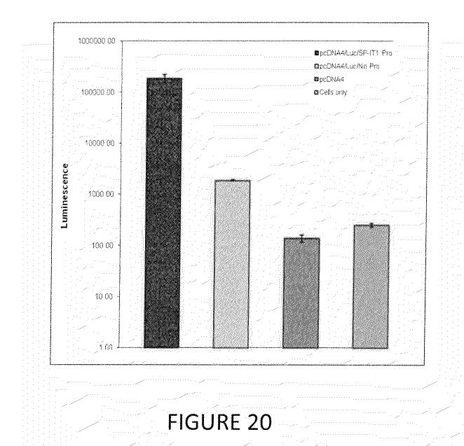

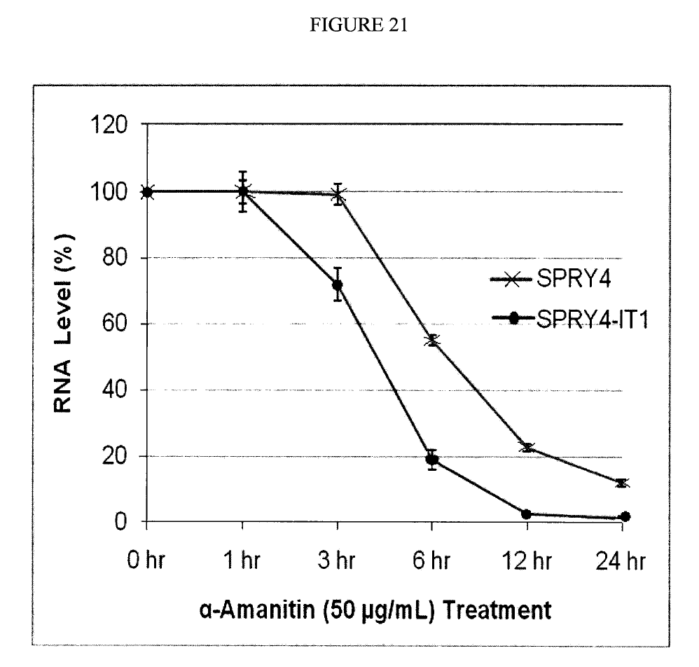

To identify the promoter elements of SPRY4-IT1, a construct was made as depicted in FIG. 19. First, the SPRY4-IT1 upstream region (1421 bp) was cloned in front of a luciferase reporter gene measured the luciferase activity. The results as shown in FIG. 20 demonstrate that the upstream sequence does contain promoter activity. Further, a vector has been constructed that contains the entire intron one (4588 bp) of the SPRY4 gene containing the entire SPRY4-IT1 gene) to determine if downstream regulatory elements are necessary for expression. A 3′ probe of SEQ ID NO:3 and a 5′ probe having the sequence GCCTTTTGGGAGGCCAAGGTAGGC (SEQ ID NO:5) were designed for RNA-FISH analysis, and results of this assay demonstrates that the 600 bp cytoplasmic version of the RNA is excised from the 743 bp full length transcript. 5′-RACE reactions (FirstChoice RLM kit, Lifetechnologies) to identify the location of the cleavage. To identify the decay rate, melanoma cells (A375) were incubated with α-amanitin, an RNA polymerase II inhibitor (irreversible inhibition in tissue culture cells at 50 μg/ml.) The expression of SPRY4-IT1 was then measured by qRT-PCR and Northern blotting using the protocols described above after 3, 6, 12, and 24 hours of treatment. For the positive control, a probe specific to mascRNA, a small RNA spliced from MALAT1 ncRNA, which has the sequence GATGCTGGTGGTTGGCACTCCTGGCATTTTCCAGGACGGGGTTGAAATCCCTGCG GCGTC (SEQ ID NO:6) and has been shown to decay during a 12 hour treatment with α-amanitin. As shown in FIG. 21, 80% of SPR4-IT1 transcript was decayed in the first three hours, which is faster than its host gene SPRY4, which has a 40% decay, for the same period in melanoma cell line A375. This indicates that downstream regulatory elements are necessary for expression.

### Protein Partners

To characterize SPRY4-IT1-interacting protein partners, RNA co-immunoprecipitation (RIP) experiments were performed to capture proteins that specifically bind to SPRY-4-IT1, and then to characterize the associated proteins by mass spectrometry (MS). A 25-bp complementary sequence to SPRY4-IT1 and having the sequence TTAATAGGCCTTGOAATCAGAAAGC (SEQ ID NO:7) was constructed utilizing a locked nucleic acid (LNA) backbone and a 5′-biotin label. This probe was used as bait to pull down SPRY-1-IT1 RNA from melanoma cell lysates, along with any associated molecules. A control probe was designed complementary to the test probe sequence and having the sequence GCTTTCTGATTCCAAGGCCTATTAA (SEQ ID NO:8). The RNA-protein complex was captured on streptavidin columns. RNA was isolated from the pull-down complexes and the amount of SPRY4-IT1 attached to the complex was verified by qRT-PCR. The RNA-protein complex was subjected to LTQ Orbitrap Velos mass spectrometry for further analysis. Table 1 depicts the candidate proteins identified by LTQ Orbitrap Velos mass spectrometry. Two of the proteins with the highest binding affinity are astacin-like metalloendopeptidase (ASTL) and phosphatidate phosphatase (LPIN2), with 372 spectral counts (indicative of protein abundance) for ASTL and 83 counts for LPIN2. Neither protein was detected in the control sample, suggesting these proteins may be relevant to the function of SPRY-1-IT1 in melanoma. This confirms that, not only does SPRY4-IT1 function with the assistance of protein partners with high binding affinity, but those proteins have been narrowed to a group delineated below in Table 1:

TABLE 1

| | Detectable Spectral |
|---|---|
| | counts from the LTQ |
| Protein | Orbitrap Velos Mass |
| Name | Spectrometer |
| GPR37 | 7 |
| RPLP1 | 2 |
| IGF2BP1 | 2 |
| RPS3 | 2 |
| RPS6 | 2 |
| LPIN2 | 83 |
| ALB | 3 |
| HNRNPCL1 | 3 |
| TRAP1 | 13 |
| TUBB2B | 11 |
| HSPE1 | 2 |
| DPYSL2 | 2 |
| RPL24 | 2 |
| IGH | 9 |
| ASTL | 372 |

### Example 10

### SPRY4-IT1 Expression in Prostate Cancer Cells

In order to confirm expression and co-localization of SPRY4-IT1 with protein partners in PC-3 prostate cancer cells, RNA-FISH assays were performed as follows: locked nucleic acid (LNA)-modified probes for human lncRNA SPRY4-IT1 having the sequence of SEQ ID NO:3 and a negative/Scramble control having the sequence of SEQ ID NO:4 were used for RNA in situ hybridization. In situ hybridization was performed using the RiboMap in situ hybridization kit (Ventana Medical Systems Inc) on a Ventana machine. The cell suspension diluted to 10,000 cells per 100 μL was pipetted into clonal rings on the autoclaved glass slides. The following day, the clonal rings were removed; slides were washed in PBS and fixed in 4% paraformaldehyde and 5% acetic acid. After acid treatment using hydrochloride-based RiboClear reagent (Ventana Medical Systems) for 10 min at 37° C., the slides were treated with the ready-to-use protease 3 reagent. The cells were hybridized with the antisense LNA riboprobe (40 nM) using RiboHybe hybridization buffer (Ventana Medical Systems) for 2 h at 58° C. after an initial denaturing prehybridization step for 4 min at 80° C. Next, the slides were subjected to a low-stringency wash with 0.1×SSC (Ventana Medical Systems) for 4 min at 60° C., and then two further washing steps with 1×SSC for 4 min at 60° C. These slides were fixed in RiboFix and counterstained with 4′-6′ diamidino-2-phenylindole (DAPI), in an antifade reagent (Ventana), fluorescein isothiocyanate (FITC), and Alexa 546. The images were acquired using a Nikon A1R VAAS laser point- and resonant-scanning confocal microscope equipped with a single photon Ar-ion laser at 60× with 4× zoom. The images of each stain were superimposed into a merged image to show co-localization.

Three sets of images resulted. In the first, images of DAPI-stained nuclei, FITC-stained SPRY4-IT1, and Alexa 546-stained Anti-L7a (to show localization of endogenous ribosomes), were superimposed. In the second, images of DAPI-stained nuclei, FITC-stained SPRY4-IT1, and Alexa 546-stained Phalloidin were superimposed. In the last set, images of DAPI-stained nuclei, FITC-stained SPRY4-IT1, and Alexa 546-stained anti-rck/p54 (associated with mRNA decay) were superimposed. The three sets show not only the cytoplasmic location of SPRY4-IT1, but also demonstrate a pronounced pattern of colocalization of SPRY4-IT1 with endogenous ribosomes and L7a, actin as shown by phalloidin, and anti-rck/p54, the latter of which is a proto-oncogene that has been shown to be overexpressed in tumor tissue and likely regulates mRNA decay. The high degree of colocalization shown in the superimposed images confirms a high likelihood of biological interaction between SPRY4-IT1 and the protein partners associated with prostate cancer.

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other physical and electronic documents.

The inventions illustratively described herein may suitably be practiced in the absence of any element or elements, limitation or limitations, not specifically disclosed herein. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

What is claimed is:

1. A method for diagnosing melanoma in a subject suspected of having melanoma comprising:
(i) assessing the expression level of SPRY4-IT1 in a biological sample obtained from the subject;
(ii) comparing the expression level of SPRY4-IT1 in the sample to the a reference expression level derived from the expression level of SPRY4-IT1 in samples obtained from subjects diagnosed as not having melanoma; and
(iii) identifying the subject as having melanoma when the expression level of SPRY4-IT1 in the sample is greater than the reference expression level or identifying the subject as not having melanoma when the expression level of SPRY4-IT1 in the sample is not greater than the reference expression level.

2. The method of claim 1, wherein the biological sample comprises skin epidermis.

3. The method of claim 1, wherein the biological sample comprises melanocytes.

4. The method of claim 1, wherein the expression level of SPRY4-IT1 is assessed by evaluating the amount of SPRY4-IT1 mRNA in the biological sample.

5. The method of claim 1, further comprising assessing a SPRY4-IT1 target and identifying the subject as having melanoma when the expression level of both SPRY4-IT1 and the SPRY4-IT1 target is increased.

6. The method of claim 5, wherein the SPRY4-IT1 target is selected from the group consisting of Ki-67, MCM2, MCM3, MCM4, MCM5, CDK1, CDC20, XIAP, Hsp60, Hsp70, and Livin.

7. The method of claim 1, further comprising assessing a SPRY4-IT1 target and identifying the subject as having melanoma when the expression level of SPRY4-IT1 is increased and the expression level of the SPRY4-IT1 target is decreased.

8. The method of claim 7, wherein the SPRY4-IT1 target is selected from the group consisting of TNFRSF25, DPP-IV, CD26, and Trail R2/DR5.

9. A method for determining the risk of a subject for developing melanoma comprising:
(i) assessing the expression level of SPRY4-IT1 in a biological sample obtained from the subject;
(ii) comparing the expression level of SPRY4-IT1 in the sample to the a reference expression level derived from the expression level of SPRY4-IT1 in samples obtained from subjects diagnosed as not having melanoma; and
(iii) identifying the subject as having increased risk of developing melanoma when the expression level of SPRY4-IT1 in the sample is greater than the reference expression level or identifying the subject as not having an increased risk of melanoma when the expression level of SPRY4-IT1 in the sample is not greater than the reference expression level.

10. The method of claim 9, wherein the biological sample comprises skin epidermis.

11. The method of claim 9, wherein the biological sample comprises melanocytes.

12. The method of claim 9, wherein the expression level of SPRY4-IT1 is assessed by evaluating the amount of SPRY4-IT1 mRNA in the biological sample.

13. The method of claim 9, further comprising assessing a SPRY4-IT1 target and identifying the subject as having an increased risk of developing melanoma when the expression level of both SPRY4-IT1 and the SPRY4-IT1 target is increased.

14. The method of claim 13, wherein the SPRY4-IT1 target is selected from the group consisting of Ki-67, MCM2, MCM3, MCM4, MCM5, CDK1, CDC20, XIAP, Hsp60, Hsp70, and Livin.

15. The method of claim 9, further comprising assessing a SPRY4-IT1 target and identifying the subject as having an increased risk of developing melanoma when the expression level of SPRY4-IT1 is increased and the expression level of the SPRY4-IT1 target is decreased.

16. The method of claim 15, wherein the SPRY4-IT1 target is selected from the group consisting of TNFRSF25, DPP-IV, CD26, and Trail R2/DR5.

17. A method for treating a patient diagnosed as having melanoma comprising administering to the patient an effective amount of a therapeutic agent that reduces SPRY4-IT1 expression.

18. The method of claim 17, wherein the SPRY4-IT1 expression is reduced in the melanoma cells.

19. The method of claim 17, wherein the SPRY4-IT1 expression is reduced by at least 50%.

20. The method of claim 17, wherein the therapeutic agent is an siRNA.

21. The method of claim 20, wherein the therapeutic agent comprises a nucleic acid comprising the sequence of SEQ ID NO: 2.

22. The method of claim 20, wherein the nucleic acid is encoded in a vector.

23. The method of claim 20, wherein the therapeutic agent is contained within a liposome.

24. A method of diagnosing prostate cancer in a subject suspected of having prostate cancer comprising:
(i) assessing the expression level of SPRY4-IT1 in a biological sample obtained from the subject;
(ii) comparing the expression level of SPRY4-IT1 in the sample to a reference expression level derived from the expression level of SPRY4-IT1 in samples obtained from subjects diagnosed as not having prostate cancer; and
(iii) identifying the subject as having prostate cancer when the expression level of SPRY4-IT1 in the sample is greater than the reference expression level or identifying the subject as not having prostate cancer when the expression level of SPRY4-IT1 in the sample is not greater than the reference expression level.

25. The method of claim 24, wherein the biological sample comprises a tissue sample.

26. The method of claim 24, wherein the expression level of SPRY4-IT1 is assessed by evaluating the amount of SPRY4-IT1 mRNA in the biological sample.

27. A method for determining the risk of a subject for developing prostate cancer comprising:
(i) assessing the expression level of SPRY4-IT1 in a biological sample obtained from the subject;
(ii) comparing the expression level of SPRY4-IT1 in the sample to the a reference expression level derived from the expression level of SPRY4-IT1 in samples obtained from subjects diagnosed as not having prostate cancer; and
(iii) identifying the subject as having increased risk of developing prostate cancer when the expression level of SPRY4-IT1 in the sample is greater than the reference expression level or identifying the subject as not having an increased risk of developing prostate cancer when the expression level of SPRY4-IT1 in the sample is not greater than the reference expression level.

28. The method of claim 27, wherein the biological sample comprises a tissue sample.

29. The method of claim 27, wherein the expression level of SPRY4-IT1 is assessed by evaluating the amount of SPRY4-IT1 mRNA in the biological sample.

30. A method for treating a patient diagnosed as having prostate cancer comprising administering to the patient an effective amount of a therapeutic agent that reduces SPRY4-IT1 expression.

31. The method of claim 30, wherein the SPRY4-IT1 expression is reduced in the prostate cancer cells.

32. The method of claim 30, wherein the SPRY4-IT1 expression is reduced by at least 50%.

33. The method of claim 30, wherein the therapeutic agent is an siRNA.

34. The method of claim 30, wherein the therapeutic agent comprises a nucleic acid comprising the sequence of SEQ ID NO:2.

35. The method of claim 34, wherein the nucleic acid is encoded in a vector.

36. The method of claim 30, wherein the therapeutic agent is contained within a liposome.
